(19) United States
(12) Patent Application Publication
(10) Pub. No.: US 2023/0346996 A1
(43) Pub. Date: Nov. 2, 2023

(54) SANITIZATION EDGE COMPUTE DEVICE

(71) Applicant: Intellisite, Ceres, CA (US)

(72) Inventors: Satyajit Banerjee, Lancaster, PA (US); Andrew Pennell, Rockingham, VT (US)

(21) Appl. No.: 17/733,778

(22) Filed: Apr. 29, 2022

(51) Int. Cl. A61L 2/20; A61L 2/10

(52) U.S. Cl. CPC A61L 2/10; A61L 2/202; C02F 1/78

(57) ABSTRACT

Sanitization edge compute devices are discussed herein. For example, an ozone generator may be controlled to output ozone in an environment to achieve a first ozone concentration level at a first time. A first decay rate of the ozone in the environment may be determined from the first ozone concentration level during a first period of time. The ozone generator may be controlled to output the ozone in the environment to a second ozone concentration level at a second time. A second decay rate of the ozone in the environment may be determined from the second ozone concentration level during a second period of time. A difference between the first decay rate and the second decay rate may be determined. An action may be performed based on the difference.

### BACKGROUND

Cleaning, disinfection, and/or sanitization technology is useful in various scenarios, such as transportation, the food industry, medical settings, public locations, homes, and the like. Without proper cleaning/disinfection/sanitization, foodborne diseases may outbreak, and fomite-based transmissions may occur. Conventionally, an enclosed space or items/objects can be cleaned and disinfected by heating, steaming, spraying, drying, salting, vacuuming, raising the pH, and/or adding chlorine. Such methods may destroy and inhibit pathogens, but there are limitations.

For example, chlorine is widely used for disinfection because it is cheap and easy to produce, store, and transport. When applied, chlorine has a residual effect and continues to perform a disinfection function (like in swimming pools). However, chlorine has some weaknesses. For example, it leaves a strong smell and can only be used in restricted quantities in food processing, such as washing fruit, greens, and vegetables. Moreover, chlorine is corrosive in higher concentrations and is limited to being applied to non-porous surfaces.

A steam autoclave is an example conventional disinfection device, which is an enclosed chamber used to remove microorganisms from items/objects. The steam autoclave uses a combination of high heat steam and pressure to achieve high levels of disinfection for items/objects such as surgical equipment. However, the conventional steam autoclave is limited to heat and moisture-tolerant materials such as stainless steel. The steam autoclave cannot be used for delicate items/objects.

Further, it is difficult to validate the cleaning/disinfection/sanitization results with the conventional methods. For example, when chlorine-based chemicals are sprayed on a kitchen counter, the user assumes that pathogens are killed, but there is no feedback verification regarding whether that is true. Though a microbiology lab can tell how much cleaning/disinfection/sanitization work has been done, that may take days.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 illustrates an example environment including one or more cleaning systems associated with one or more monitored locations in accordance with examples of the disclosure.

FIG. 2 illustrates an example environment that is usable to implement the techniques and systems described herein.

FIG. 3 illustrates an example cleaning system in accordance with examples of the disclosure.

FIG. 4 illustrates an example environment where a cleaning system includes cleaning devices deployed at different monitored locations in accordance with examples of the disclosure.

FIG. 5 illustrates an example environment where a cleaning system is placed inside a monitored location in accordance with examples of the disclosure.

FIG. 6 illustrates an example cleaning system with an internal chamber to clean, disinfect, and/or sanitize items/objects and/or environments in accordance with examples of the disclosure.

FIG. 7 illustrates an example graph of ozone concentration versus time in accordance with examples of the disclosure.

FIG. 8 illustrates an example graph representing ozone concentration curves as straight lines.

FIG. 9 illustrates an example environment including one or more cleaning systems and one or more remote computing devices in accordance with examples of the disclosure.

FIG. 10 illustrates a graphical user interface (GUI) illustrating a report/alert page for a monitored location in accordance with examples of the disclosure.

FIG. 11 illustrates another graphical user interface (GUI) illustrating a report/alert page for a monitored location in accordance with examples of the disclosure.

FIG. 12 illustrates an example cleaning, disinfection, and/or sanitization process 1200 in accordance with examples of the disclosure.

FIG. 13 illustrates an example cleaning, disinfection, and/or sanitization process in accordance with examples of the disclosure.

FIG. 14 illustrates an example cleaning, disinfection, and/or sanitization process in accordance with examples of the disclosure.

FIG. 15 illustrates an example cleaning, disinfection, and/or sanitization process in accordance with examples of the disclosure.

### DETAILED DESCRIPTION

This disclosure is directed to techniques for cleaning systems and methods. For example, in the context of cleaning and/or disinfecting vehicles, shipping containers, food processing equipment, enclosed space in medical settings, waiting rooms, homes, items/objects within an enclosed space, the cleaning methods and systems as described herein use non-thermal mechanisms - ozone generators and UVC light units, controlled by computing devices - to conduct cleaning, disinfection, and/or sanitization. UVC light acts quickly on readily exposed surfaces, whereas the ozone penetrates deeper, reaching hidden surfaces and crevices. Ozone generators and UVC light units can work individually, in combination, or alternately to achieve cleaning/disinfection/sanitization results. Some examples provide an edge computing device, which may perform the algorithm for generating ozone, determining the decay rate of the ozone, and determining a difference between decay rates. The edge computing device may operate standalone or in conjunction with a central computing device. Some examples provide central computing which includes a cloud unit that receives data from various edge computing devices and parameters associated with disinfecting cycles and sending instructions to control the edge computing devices. Some examples provide a system for reporting and alerting, where the central computing device receives data from the edge computing devices and makes suggestions/alerts to send to customers/users. Various examples of the cleaning systems and methods with respect to the techniques are provided herein.

Techniques described herein use sensor feedback to maintain a safe and more effective ozone concentration. Moreover, ozone is a stronger oxidizer than chlorine and it leaves no residual smell. Pathogens may be removed significantly without adversely affecting the commodity. The cleaning process may be improved.

The techniques described herein are environment-friendly. Ozone degrades back into oxygen and UVC dissipates naturally. The cleaning processes with ozone and UVC only consume electricity and air. In some examples, a system including a humidifier unit may further consume water to humidify the cleaning environment. Additionally, the cleaning systems and methods can be used to augment wastewater processing with the incorporation of aqueous ozone.

The techniques described herein may use artificial intelligence and/or machine learning techniques. Each time the cleaning system runs anywhere in the world, data of the parameters used, and the outcome reduction in pathogen levels from lab results may be transmitted to the central computing device. The data may be fed a machine learned model that is constantly learning how to get the deepest disinfection in the shortest time possible under different circumstances. These findings may be fed back to cleaning systems at different monitored locations to constantly improve the outcome. The machine learned model may also uncover trends from the received data, which may be relayed back to each customer.

The techniques described herein can be implemented in a number of ways. Example implementations are provided below with reference to the following figures. Although discussed in the context of cleaning, disinfection, and/or sanitization of enclosed spaces and/or items/objects within enclosed spaces, the methods, apparatuses, and systems described herein can be applied to a variety of scenarios, and are not limited to enclosed spaces and/or items/objects within enclosed spaces. In another example, the techniques can be utilized in an aviation or nautical context. Additionally, the techniques described herein can be used with real data (e.g., captured using the sensor(s)), simulated data (e.g., generated by a simulator), or any combination of the two.

Definitions of terminologies are provided herein. It should be understood that the definitions are used to help an ordinary person skilled in the art understand this disclosure rather than limiting the scope thereof.

Adenosine triphosphate (ATP) - a molecular compound found in all living tissue because it forms the basis of energy creation. An ATP sensor reads the level of ATP as Relative Light Units (RLUs).

Clean In Place / Clean Out of Place (CIP/COP) - Some industrial equipment can only be sanitized where it is, because it is too big and bulky. This is CIP. When components can be dismantled and cleaned elsewhere with more care and attention, such as in an autoclave, it is called COP.

Non-thermal autoclave - An enclosure that achieves a level of disinfection without the use of heat or steam. The non-thermal autoclave can be used to disinfect many items/objects that are not heat or moisture resistant.

Concentration of ozone - ozone concentration is presented as parts per million (ppm). The context is important though, because 10 ppm of gaseous ozone (in the air) is different from 10 ppm of aqueous ozone.

Density of Ozone - Ozone’s density is around 2.14 kg / m³ at the standard air pressure, temperature, and humidity, compared to that of air, 1.225 kg /m³.

Electron ionization - the process by which highly energized electrons are used to bombard molecules to create ions. In the case of ozonation, the process splits diatomic oxygen into ions, which then recombine with other diatomic oxygen molecules, to form triatomic oxygen molecules.

Oxidation - oxidation occurs when an atom, molecule, or ion loses one or more electrons as a result of a chemical reaction. The opposite is reduction, which happens when electrons are gained. Two strong oxidizing agents are chlorine and ozone, which is why they can be used in disinfectants.

Ozone destructor - a device used to rapidly remove ozone by converting it into oxygen.

Disinfection - a significant reduction in the number of pathogens.

UVC light - UVC is electromagnetic radiation in the 200-280 nm range of wavelengths, and is an effective germicide. It is absorbed by conventional glass and acrylic but is propagated by quartz glass. Ultraviolet light is divided into 4 groups of wavelengths, from the longest to the shortest wavelength (the same as from the lowest to the highest frequency): UVA (315-400 nm), UVB (280-315 nm), UVC (200-280 nm), and VUV or vacuum UV (100-200 nm).

Irradiation - The rate at which UV light energy lands on a given unit area, denoted by W/m².

Fluence - The cumulative amount of UV light energy lands on a given unit area, denoted by J/m².

Pathogen - any disease-causing microorganism, including viruses, bacteria, fungi, and protozoa.

Food-borne pathogens - parasites or bacteria that are found in food or beverages. They are commonly acquired through ingestion of contaminated material, causing food poisoning. Common examples include E. coli, Salmonella, Listeria, Cyclospora, Hepatitis A, Clostridium, and so on.

Bacteria - a diverse family of single-celled organisms that play a critical role in the life cycle. Some are pathogenic to humans.

Biofilm - Biofilms are a collective of one or more types of microorganisms that can grow on many different surfaces. The extracellular material protects the microbes within and is sometimes slimy.

Virus - a non-living protein-encapsulated pathogen that reproduces by inserting its DNA or RNA into a living host cell, often destroying it in the process. They are so small they cannot be seen under a microscope. They are inactive outside a living cell, and therefore have no dependency on organic matter, the presence of which is often measured using ATP luminometers.

Fungus - a member of the massive Fungi family of organisms that includes mold, fungus, and yeast.

Mold - a type of Fungi that thrives in dark and damp places and appears as a furry growth.

Colony Forming Units (CFUs) - CFU is considered the result of a single bacterium reproducing in a petri dish to form a visible and countable unit. The total viable count is the CFU / ml. The number of CFUs depends on the number of contaminants in the sample. Serial dilutions are used so that one of the dishes will have between 30 and 100 CFUs, which is considered an acceptable number to count.

Log reduction - a ten-fold reduction in pathogenic density, typically measured as CFUs per unit volume or unit mass. For example, a 1 log reduction is 10^1, so the final count is one tenth of the original, or a reduction of 90%. 2 log is 10^2, or one hundredth, or 99%. 3 log is a thousandth, or 99.9%, and so on. In the instance of a pathogenic density around 10^5 CFU/ml, a 6-log reduction would bring that down to less than 1.

Serial dilutions - A solution is repeatedly diluted by a solvent, typically distilled water. For counting CFUs, ten-fold dilutions are commonly used.

Artificial Intelligence of Things (AIoT) is the combination of Artificial intelligence technologies with the Internet of things infrastructure to achieve more efficient IoT operations, improve human-machine interactions and enhance data management and analytics.

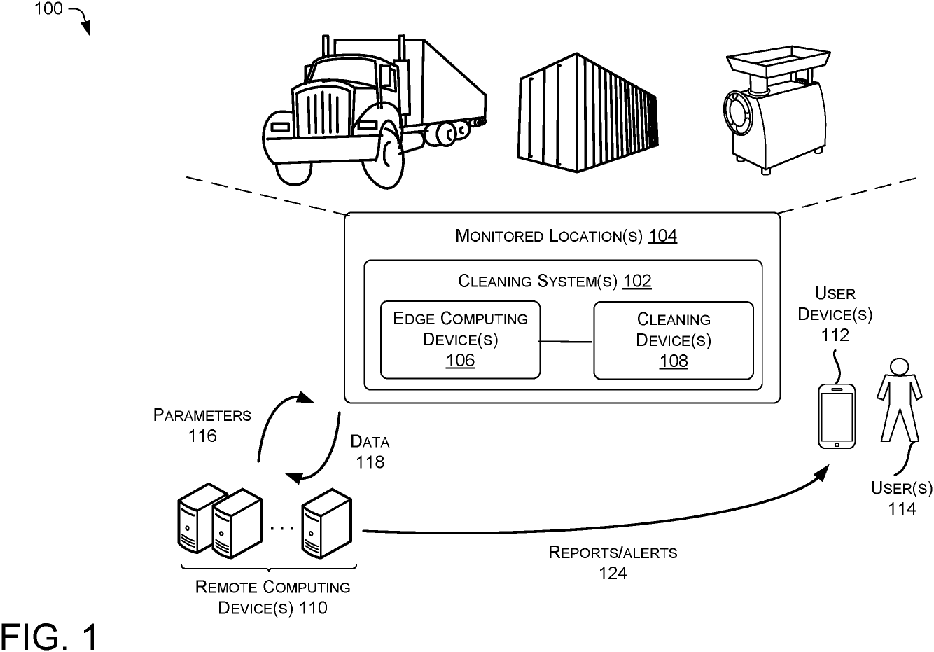

FIG. 1 illustrates an example environment 100 including one or more cleaning systems 102 associated with one or more monitored locations 104, in accordance with examples of the disclosure. As described herein, the monitored location(s) 104 may include, but is not limited to, transportation instruments (e.g., school buses, trucks, shipping containers, and the like), farm settings (e.g., incubators, hatchery rooms, and the like), medical settings (e.g., operation rooms, waiting rooms, healthcare clinic rooms, ambulances, and the like), public locations (e.g., cinemas, restaurants, offices, stores, hotels, clubhouses, and the like), home settings (e.g., dining rooms, kitchens, kitchen appliances (e.g., a dishwasher), bedrooms, and the like), and the like. Additionally or alternatively, the monitored location(s) 104 may include things or items/objects to be cleaned/disinfected/sanitized such as cargos (e.g., goods carried in trucks, vans, airplanes, trains, ships, and the like), food processing equipment (e.g., meat grinders, cutting boards, utensils, and the like), food (e.g., fruit, vegetables, chilling poultry, and the like), surgical tools (e.g., scissors, surgical blades, knives and scalpels, and the like), furniture (e.g., tables, chairs, sofas, carpets, curtains, and the like), decorations (e.g., paintings, plants, ornaments, and the like), and the like.

The cleaning system(s) 102 may be placed at the monitored location(s) 104 within an enclosed space to be cleaned/disinfected/sanitized. Additionally or alternatively, the cleaning system(s) 102 may include an enclosed space to contain the items/objects to be cleaned/disinfected/sanitized. The cleaning system(s) 102 may include one or more edge computing devices 106 and one or cleaning devices 108. In some examples, the cleaning devices(s) 108 may be implemented as biosecurity autoclaves, cleaning robots, etc. In some examples, the cleaning device(s) 108 may perform cleaning/disinfection/sanitization utilizing ozone and/or UVC, controlled by the edge computing device(s) 106. In some examples, the cleaning device(s) 108 may further include one or more humidifiers configured to inject moisture into the monitored location(s) 104, which can be controlled by the edge computing device(s) 106. In some examples, the cleaning device(s) 108 may further include one or more destructors, which can convert the ozone back to oxygen. In some examples, the cleaning device(s) 108 may use information provided by the AIoT. In some examples, the cleaning device(s) 108 may be configured to remove pathogens (e.g., bacteria, fungi, spores, viruses, protozoa, and the like) from spaces, surfaces, items/objects, and so on.

The cleaning system(s) 102 may communicate with one or more remote computing devices 110computing devices 110, which in turn can communicate with one or more user devices 108 associated with one or more users 114. In some examples, the remote computing device(s) 110 may send parameters 116 to the edge computing device(s) 106 of the cleaning system(s) 102, and the edge computing device(s) 106 of the cleaning system(s) 102 may send data 118 to the remote computing device(s) 110. In some examples, the remote computing device(s) 110 may send reports/alerts 124 to the user device(s) 112 associated with the user(s) 114. In some examples, the remote computing device(s) 110 may communicate with the cleaning system(s) 102 and the user device(s) 112 via wired or wireless connections.

In some examples, the cleaning system(s) 102 may provide solutions to Clean In Place (CIP) and/or Out of Place (COP) scenarios. For example, the cleaning system(s) 102 in custom sizes, is a good example of COP for mechanical parts like meat grinding components, cutting boards, and utensils. Using ozonolysis and UVC irradiation on cycling water is a good example of CIP, for things like washing fruit and vegetables, or chilling poultry.

As an example, the cleaning system(s) 102 with ozone and UVC may perform cleaning/disinfection/sanitization in rooms. In some examples, cleaning system(s) 102 can clean/disinfect the rooms with high concentrations of ozone before use, and then gently maintain a safe level of ozone using ultra-low background concentrations when the rooms are in use. When the cleaning/disinfection/sanitization process of rooms is going on, the rooms may to be sealed off, so that the ozone gas does not leak. Rooms typically circulate air, defined by Air Changes per Hour (ACH). It may be hard to ozonate a room if the air is constantly being replaced every few minutes. Windows also need to be covered to avoid the risk of people looking at the UVC light, because it may damage eyesight.

In some examples, multiple fixed or portable ozone generators, UVC light units, humidifiers, and/or destructors may be used in any combination. The ozone generators, UVC light units, humidifiers, and/or destructors could be scattered or otherwise placed independently throughout the room. The UVC light units may be placed individually to account for the relatively short range of effectiveness. For example, in the context of sanitizing a medical setting, such as a shared dialysis room, a UVC light unit may be placed in front of each patient chair (or other locations), as such areas are high-touch areas where healthcare associated infections may originate.

In some examples, a cleaning environment may include materials inside for sanitization. Materials that are susceptible to damage from the ozone may be removed from the room before cleaning/disinfection/sanitization. An example list of materials is given below.

Ozone safe materials include: 316 stainless steel, aluminum, ceramic, polycarbonate, polyurethane, polytetrafluoroethylene (PTFE), polyurethane, silicone, titanium, and the like.

Materials that can cope with some exposure to ozone include: ABS plastic, acrylic, low-density polyethylene (LDPE), polyvinyl chloride (PVC), polyacrylate, polyethylene, and the like.

More sensitive materials that can only handle brief exposure to ozone include: cast iron, galvanized steel, Neoprene, and the like.

Materials that should be removed from the room during cleaning/disinfection/sanitization include: nitrile, natural rubber, nylon, mild steel, zinc, and the like.

It should be understood that the above list is not exhaustive. There may be other materials in each category.

As an example, the cleaning system(s) 102 with ozone and UVC may perform cleaning/disinfection/sanitization in transportation vehicles (e.g., a bus, a train compartment, and the like). The transportation vehicle has some similarities to a room, but with its own characteristic. For example, a room can have a permanent UVC and ozone installation, whereas a transportation vehicle may use portable units. For example, buses and trains are long and narrow, which presents some challenges. In some examples, when an area in a bus is under exposure, the UVC light source may be intensified or additional UVC light units may be added so that each area of the bus could be correctly exposed. When an area is over-exposed, materials may be damaged, so the UVC light source may be dimmed or removed. The layout of UVC light sources may be configured to achieve optical results. In some examples, an air circulation fan may be used to help dissipate the ozone throughout the internal space of the transportation vehicle.

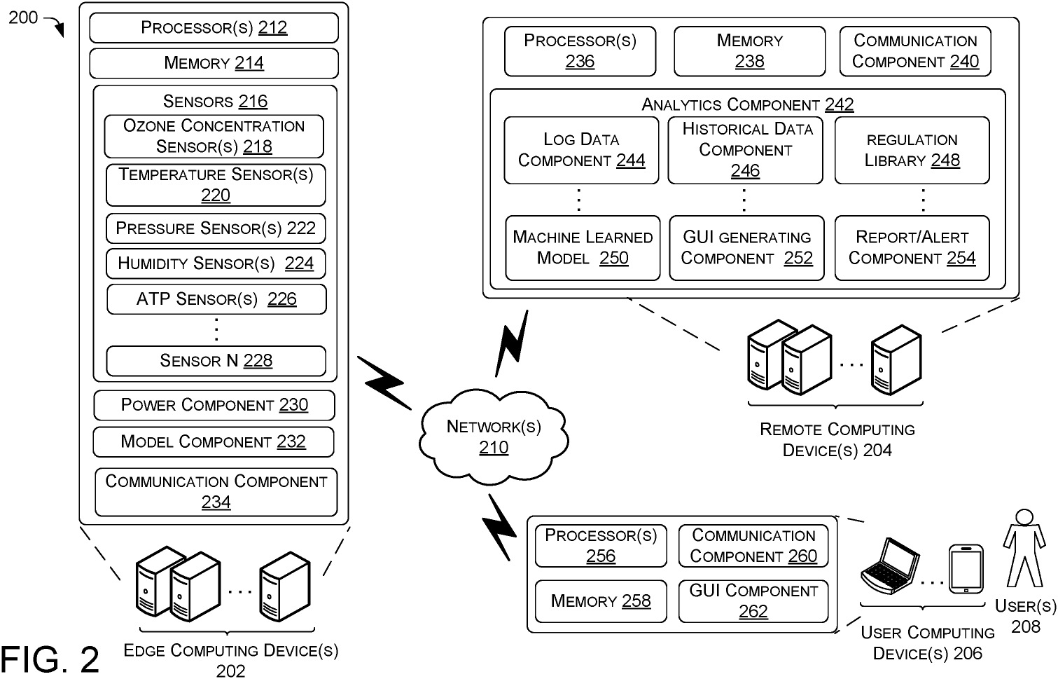

FIG. 2 illustrates an example environment 200 that is usable to implement the techniques and systems described herein. The environment 200 includes a plurality of devices such as edge computing device(s) 202 configured to gather and process data described herein. The environment 200 also includes one or more remote computing device(s) 204 that can further provide processing and analytics. The remote computing device(s) 204 is configured to communicate alerts, reports, analytics, recommendations, instructions, graphical user interfaces, etc., to the user computing device(s) 206 associated with the user(s) 208. In various examples, the edge computing device(s) 202, the remote computing device(s) 204, and the user computing device(s) 206 can communicate wired or wirelessly via one or more networks 210.

The edge computing device(s) 202 can individually include, but are not limited to, any one of a variety of devices, including portable devices or stationary devices. For instance, a device can comprise a data logger, an embedded system, a programmable logic controller, a sensor, a monitoring device, a smart phone, a mobile phone, a personal digital assistant (PDA), a laptop computer, a desktop computer, a tablet computer, a portable computer, a server computer, a wearable device, or any other electronic device. In various examples, the edge computing device(s) 202 can correspond to the edge computing device(s) 102 of FIG. 1.

The edge computing device(s) 202 can include one or more processor(s) 212 and memory 214. The processor(s) 212 can be a single processing unit or a number of units, each of which could include multiple different processing units. The processor(s) 212 can include a microprocessor, a microcomputer, a microcontroller, a digital signal processor, a central processing unit (CPU), a graphics processing unit (GPU), a security processor, etc. Alternatively, or in addition, some or all of the techniques described herein can be performed, at least in part, by one or more hardware logic components. For example, and without limitation, illustrative types of hardware logic components that can be used include a Field-Programmable Gate Array (FPGA), an Application-Specific Integrated Circuit (ASIC), an Application-Specific Standard Products (ASSP), a state machine, a Complex Programmable Logic Device (CPLD), pulse counters, resistor/coil readers, other logic circuitry, a system on chip (SoC), and/or any other devices that perform operations based on instructions. Among other capabilities, the processor(s) 212 can be configured to fetch and execute computer-readable instructions stored in the memory.

The memory 214 can include one or a combination of computer-readable media. As used herein, “computer-readable media” includes computer storage media and communication media.

Computer storage media includes volatile and non-volatile, removable and non-removable media implemented in any method or technology for storage of information, such as computer-readable instructions, data structures, program modules, or other data. Computer storage media includes, but is not limited to, Phase Change Memory (PRAM), Static Random-Access Memory (SRAM), Dynamic Random-Access Memory (DRAM), other types of Random-Access Memory (RAM), Read Only Memory (ROM), Electrically Erasable Programmable ROM (EEPROM), flash memory or other memory technology, Compact Disk ROM (CD-ROM), Digital Versatile Disks (DVD) or other optical storage, magnetic cassettes, magnetic tape, magnetic disk storage or other magnetic storage devices, or any other medium that can be used to store information for access by a computing device.

In contrast, communication media includes computer-readable instructions, data structures, program modules, or other data in a modulated data signal, such as a carrier wave. As defined herein, computer storage media does not include communication media. In some examples, non-transitory computer-readable media does not include communication media.

The memory 214 can include an operating system configured to manage hardware and services within and coupled to a device for the benefit of other modules, components, and devices. In some examples, the one or more edge computing device(s) 202 can include one or more servers or other computing devices that operate within a network service (e.g., a cloud service), or can form a mesh network, etc. The network(s) 210 can include the Internet, a Mobile Telephone Network (MTN), Wi-Fi, a cellular network, a mesh network, a Local Area Network (LAN), a Wide Area Network (WAN), a Virtual LAN (VLAN), a private network, and/or other various wired or wireless communication technologies.

The techniques discussed above can be implemented in hardware, software, or a combination thereof. In the context of software, operations represent computer-executable instructions stored on one or more computer-readable storage media that, when executed by one or more processors, configure a device to perform the recited operations. Generally, computer-executable instructions include routines, programs, objects, components, data structures, and the like that perform particular functions or implement particular abstract data types.

The edge computing device(s) 202 can include one or more sensor(s) 216, including but not limited to, ozone concentration sensor(s) 218, temperature sensor(s) 220, pressure sensor(s) 222, humidity sensor(s) 224, ATP sensor(s) 226, and/or sensor N 228. The sensor(s) 216 can continuously or periodically monitor data at any interval, or upon request. In some examples, the edge computing device(s) 202 can include one or more expansion ports (e.g., as sensor N 228) to receive additional sensors or input from additional monitoring systems, such as individual appliances or “smart” devices. In some examples, the edge computing device(s) 202 may monitor lighting, building occupancy, building security, environmental factors (e.g., indoor/outdoor weather, temperature, wind, sun, rain, humidity, pressure, etc.), or the like. In some examples, one or more inputs and/or sensor(s) 216 can be isolated to protect the edge computing device(s) 202 from receiving damaging inputs. In some examples, the edge computing device(s) 202 can timestamp each pulse or input received by the sensor(s) 216. That is to say, each data point monitored, received, and/or transmitted by the edge computing device(s) 202 can have an associated timestamp for the generation time of the data.

The edge computing device(s) 202 can also include a power component 230 that receives power from a network such as a power grid, and can also include one or more uninterruptable power supplies (UPS) to power the edge computing device(s) 202 when power is interrupted. For example, the power component 230 can include a timer that determines a duration of time when power is absent and can shut down the edge computing device(s) 202 when the duration is beyond a threshold, without crashing, damaging, or losing data of the edge computing device(s) 202. Further, the power component 230 can monitor a power supply while the edge computing device(s) 202 is in a powered-down state and can restart the device when power is restored. In some examples, the power component 230 can send an error message when a power outage is detected. In some examples, the power component 230 can include one or more power filters to filter an incoming power supply to reduce a number of spurious or false counts received and/or generated by the sensor(s) 216.

The edge computing device(s) 202 can also include a model component 232. In some examples, in the event of an interruption to network connectivity, the edge computing device(s) 202 may continue to function autonomously using intelligence built in model component 232. In some examples, the model component 232 can include any models, algorithms, heuristics, and/or machine learning algorithms. For example, model component 232 can be implemented as a neural network. As described herein, an exemplary neural network is a biologically inspired algorithm which passes input data through a series of connected layers to produce an output. Each layer in a neural network can also comprise another neural network, or can comprise any number of layers (whether convolutional or not). As can be understood in the context of this disclosure, a neural network can utilize machine learning, which can refer to a broad class of such algorithms in which an output is generated based on learned parameters.

Although discussed in the context of neural networks, any type of machine learning can be used consistent with this disclosure. For example, machine learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc.

The edge computing device(s) 202 can include a communication component 234 to communicate with other edge computing device(s) (e.g., in mesh network) and/or to communicate via the network(s) 210. For example, the communication component 234 can perform compression, encryption, and/or formatting of the data received and/or generated by the sensor(s) 216. In some examples, the communication component 234 can transmit data using one or more protocols or languages, such as an extensible markup language (XML), Modbus, HTTP, HTTPS, USB, etc.

The remote computing device(s) 204 can include one or more processor(s) 236, a memory 238, and a communication module 240, each of which can be implemented similar to the processor(s) 212, the memory 214, and/or the communication component 234 of the edge computing device(s) 202.

In some examples, the remote computing device(s) 204 can include an analytics module 242, including one or modules such as a log data component 244, a historical data component 246, a regulation library 248, a machine learned model 250, a graphical user interface (GUI) generating component 252, and/or a report/alert component 254.

The analytics module 242 can receive data from the edge computing device(s) 202 and can store the data in the log data component 244. In some examples, the data may include log data indicative of parameters and ozone decay rate data associated with monitored locations. In some examples, the data may indicate the internal temperature of the monitored location, the internal pressure of the monitored location, the internal humidity of the monitored location, the dimension of the monitored location, the external temperature of the monitored location, the external pressure of the monitored location, external humidity of the monitored location, materials to be disinfected, and the like. In some examples, the data may include the weather (temperature, wind, daytime, nighttime, rain, etc.) associated with the monitored location or the like. In some examples, the data may include anonymized data. In some examples, the log data component 244 can store the timestamp of the data input from the edge computing device(s) 202.

In some examples, analytics module 242 can receive historical data and store the historical data in the historical data component 246. In some examples, the historical data can include past data for one or more monitored locations, past data for similar locations (e.g., similar vehicles, similarly situated rooms, similar items/objects, similar types of equipment, similar materials, and the like), and the like. In some examples, the historical data can be used to determine whether a current parameter is normal or not. For example, a clean signature can be used to determine whether the monitored location is clean or not.

The analytics module 242 can include a regulation library 248 which includes regulations such as environmental pollution regulations, safety regulations, hygiene standards in different industries, local regulations associated with a particular country or district, and the like. Regulatory bodies such as the Food and Drug Administration (FDA), Environmental Protection Agency EPA, U.S. Department of Agriculture (USDA), and the like may be able to make the rules/regulations. In some examples, the analytics module 242 can determine whether the cleaning/disinfection/sanitization process complies with regulations.

The analytics module 242 can include a machine learned model 250. In some examples, the machine learned model 250 can be configured and/or trained to determine how to refine the cleaning/disinfection/sanitization process. In some examples, the machine learned model 250 can be configured and/or trained to determine control parameters, which may be sent to the edge computing device(s) 202 via the network(s) 210. In some examples, the control parameters may include an ozone concentration level to pulse up to, an ozone concentration level to let the ozone concentration to fall to, an order of emitting UVC or ozone at a monitored location, a length of time of emitting the UVC, a humidity level to control a humidifier unit, an order of increasing humidity (e.g., a humidity profile over time), a maximum or minimum humidity level for a cleaning environment, an expected humidity level, an ozone concentration level for setting for a particular monitored location, an expected ozone decay rate for a particular monitored location when clean, and the like. In some examples, the analytics module 242 can compare current data to historical data associated with a single monitored location, or compare data between a plurality of monitored locations. In some examples, the analytics module 242 can correlate the log data and the historical data associated with the same monitored location. In some examples, the analytics module 242 can correlate the data associated with similar locations (e.g., similar vehicles, similarly situated rooms, similar items/objects, similar types of equipment, similar materials, and the like). In some examples, the analytics module 242 can receive one or more signatures indicating a state of the process, such as a clean signature, an error signature, an in-progress signature, and the like. For example, one or more signatures may be input to the machine learned model 250, such that the machine learned model 250 can output information indicating a state of the process, such as a clean state, an error state, an in-progress state, and the like. In some examples, the analytics module 242 can determine an abnormal event such as the disinfection cycle of the monitored location being too long. For example, the abnormal event may be determined by the machine learned model 250.

In some examples, the machine learned model 250 can include any models, algorithms, and/or machine learning algorithms. For example, the machine learned model 250 can be implemented as a neural network, as discussed above. As described herein, an exemplary neural network is a biologically inspired algorithm which passes input data through a series of connected layers to produce an output. Each layer in a neural network can also comprise another neural network, or can comprise any number of layers (whether convolutional or not). As can be understood in the context of this disclosure, a neural network can utilize machine learning, which can refer to a broad class of such algorithms in which an output is generated based on learned parameters.

The analytics module 242 can include a GUI generating component 252 which is configured to generate data to be displayed via GUIs. The analytics module 242 may send the data to be displayed to the user computing device(s) 206 via the network(s) 210. In some examples, the data to be displayed may include information indicating a state of the monitored location (e.g., a clean state, an error state, an in-progress state, and the like). In some examples, the data to be displayed may include information indicating the internal temperature of the monitored location, the internal pressure of the monitored location, internal humidity of the monitored location, dimension of the monitored location, the external temperature of the monitored location, the external pressure of the monitored location, external humidity of the monitored location, or materials to be disinfected. In some examples, the data may include the weather (temperature, wind, daytime, nighttime, rain, etc.) associated with the monitored location or the like.

The analytics module 242 can include a report/alert component 254 to generate one or more reports, alerts, and/or recommendations. In some examples, the report/alert component 254 may provide reports and/or alerts to be sent to the user computing device(s) 206 associated with the user(s) 208 in real-time, near real-time, or in an operationally relevant manner. In some examples, the report/alert may include an abnormal event associated with a monitored location, an instruction related to the abnormal event, and so on. In some examples, the report/alert may include an instruction such as further checking/inspecting the monitored location, and so on. In some examples, the report/alert may include a recommendation to further clean/disinfect/sanitize the monitored location or objects in the monitored location. In some examples, if it is taking longer to run a disinfection cycle when nothing else has changed, including temperature, humidity, and air quality, it suggests that there is an increase in something that is currently not being looked for. An investigation is needed to identify the root cause - pathogenic or not.

The user computing device 206 can include one or more processor(s) 256, a memory 258, and a communication module 260 each of which can be implemented similar to the processor(s) 212, the memory 214, and/or the communication component 234 of the edge computing device(s) 202.

The user computing device 206 can include a GUI component 262 to display or otherwise present graphical user interfaces at the user computing device 206. In some examples, the user computing device 206 may receive the data to be displayed from the remote computing device(s) 204. In some examples, the GUI component 262 may display information indicating a state of the monitored location (e.g., a clean state, an error state, an in-progress state, and the like). In some examples, the GUI component 262 may display information indicating the internal temperature of the monitored location, the internal pressure of the monitored location, internal humidity of the monitored location, dimension of the monitored location, the external temperature of the monitored location, the external pressure of the monitored location, external humidity of the monitored location, or materials to be disinfected. In some examples, the data may include the weather (temperature, wind, daytime, nighttime, rain, etc.) associated with the monitored location or the like. In some examples, the GUI component 262 may display a report/alert to a user detailing an abnormal event associated with a monitored location, an instruction related to the abnormal event, and so on. For example, the GUI component 262 may display an instruction such as further checking/inspecting the monitored location, and so on.

The environment 200 also includes one or more users 208 to employ the user computing device 206. One or more users 208 can interact with the user computing device 206 to perform a variety of operations. In some examples, the users 208 may review the reports/alerts generated by the remote computing device(s) 204 and implement any recommended changes to the monitored location and/or follow the instructions like checking/inspecting the monitored location, and the like.

The operations of the edge computing device(s) 202, the remote computing devices(s) 204, and the user computing device(s) 206 are further provided in connection with the various figures of this disclosure. Further, any of the functions or operations provided by one component can be provided by any other component discussed herein. That is, features of the edge computing device 202 can be provided by the remote computing device 204 (and/or the user computing device 206), and vice versa.

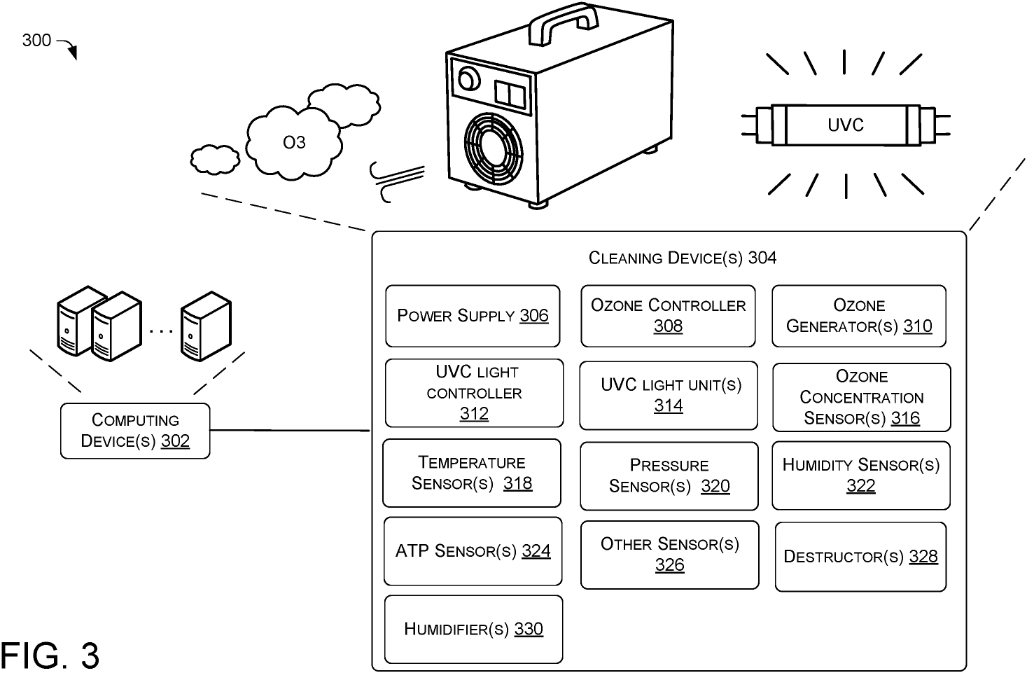

FIG. 3 illustrates an example cleaning system 300 in accordance with examples of the disclosure. The cleaning system 300 includes a plurality of devices such as computing device(s) 302 configured to gather and process data described herein, and cleaning device(s) 304 configured to perform cleaning/disinfection/sanitization. In various examples, the computing device(s) 302 and cleaning device(s) 304 can communicate wired or wirelessly. In various examples, the computing device(s) 302 can correspond to the edge computing device(s) 102 of FIG. 1 and the edge computing device(s) 202 of FIG. 2. The cleaning device(s) 304 can correspond to the cleaning device(s) 108 of FIG. 1.

The cleaning device(s) 304 can include a power supply 306 that receives power from a network such as a power grid. In some examples, the power supply 306 can include protection units to protect the cleaning device(s) 304 from surge current, overvoltage, overcurrent, leakage, and the like.

The cleaning device(s) 304 can include an ozone controller 308 that controls the ozone generator(s) 310. The ozone generator(s) 310 can generate ozone from air or oxygen. In some examples, the ozone generator(s) 310 may take the ambient air, converting the oxygen content into ozone. In some examples, an air pump (not shown) may pass ambient air through a dehumidifier (not shown) to improve the ozonation efficiency. The ozone generator(s) 310 may take the dried air, converting the oxygen content into ozone. The ozone generator(s) 310 may inject/deliver the ozone into the monitored location. In some examples, ozone concentration sensor(s) 316 may track the ozone concentrations within the monitored location so that the ozone generator(s) 310 can pause and resume the ozonation with micro-doses to maintain the ozone concentration inside the monitored location.

Ozone is an unstable and highly reactive molecule consisting of three atoms of oxygen. Ozone can also be called trioxygen or triatomic oxygen. Formula 1 illustrates the structure of an ozone molecule.

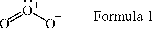

Ozone formation is a two-step process. First, an O₂ molecule is split into two unstable oxygen atoms (Formula 2). Second, one of these atoms attaches to an O₂ molecule to form O₃ (Formula 3). Ozone generators can generate ozone using such an ozone formation process.

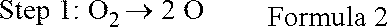

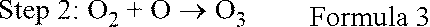

On the other hand, ozone may decay. There is a two-step decomposition process of ozone, converting ozone back to oxygen. First, a single-bonded unstable oxygen atom breaks away from the O₃ to generate O₂ and an oxygen atom (Formula 4). Second, the single atom (and maybe quite slowly) meets another ozone molecule, splitting it into 2 dioxygen molecules (Formula 5). This process may happen naturally or with a catalyst.

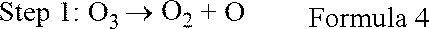

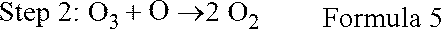

The two-step decomposition process of ozone may be significant in the cleaning/disinfection/sanitization. As the O3 molecule approaches a target molecule (e.g., proteins on a cell membrane), the unstable oxygen atom breaks away from the O3 to create O2 and an oxygen atom (Formula 4). Next, the oxygen atom steals two electrons from the target molecule in a process called oxidation. Oxidation is the loss of electrons during a reaction by a molecule, atom, or ion. The kinetics for pathogen inactivation caused by ozone may vary based on where the target is in its vegetative state, such as encapsulated or contained in a spore. Usually, the pathogen inactivation may involve oxidative reactions on the cell walls of pathogens. For example, oxidation may damage the protective membranes of microorganisms, causing the microorganisms’ innards to spill out, resulting in inactivation. Viruses are not alive, but ozone may disturb the viral envelope and cause the inactivation of the viruses. For example, ozone can cause the inactivation of SARS-CoV-2.

The ozone generator(s) 310 may inj ect/deliver the ozone into the monitored location at a rate in grams per hour (g/hr), for example, 1 g/hr, 5 g/hr, 10 g/hr, 20 g/hr, and the like. The efficiency of the ozone generator(s) 310 depends on at least one of the temperature of the incoming air (the cooler the better), the humidity of the incoming air (the drier the better), or the oxygen concentration in the incoming air (oxygen-enriched air is better). In some examples, the ozone generator(s) 310 may not operate at 100% efficiency.

The time to reach a target ozone concentration in an enclosed space of the monitored location can be calculated at least based on the ozone generation rate, the size of the enclosed space, and the target concentration level of ozone. In some examples, the enclosed space may be in a given disinfected chamber that has nothing for ozone to react with. For example, Table 1 below shows the time (in minutes) to achieve 1-25 ppm of ozone in an 18 cubic meter enclosed space with four different size ozone generators running at 40% efficiency.

TABLE 1

Time to Reach a Particular Concentration Level of Ozone

| Concentration Level of Ozone (ppm) | Ozone Generation Rate | | | |
|---|---|---|---|---|
| | 1 g/hr | 5 g/hr | 10 g/hr | 20 g/hr |
| 1 | 5.8 mins | 1.2 | 0.6 | 0.3 |
| 2 | 11.6 | 2.3 | 1.2 | 0.6 |
| 3 | 17.5 | 3.5 | 1.7 | 0.9 |
| 4 | 23.1 | 4.6 | 2.3 | 1.2 |
| 5 | 28.9 | 5.8 | 2.9 | 1.4 |
| 6 | 34.7 | 6.9 | 3.5 | 1.7 |
| 7 | 40.4 | 8.1 | 4.0 | 2.0 |
| 8 | 46.2 | 9.2 | 4.6 | 2.3 |
| 9 | 52.0 | 10.4 | 5.2 | 2.6 |
| 10 | 57.8 | 11.6 | 5.8 | 2.9 |
| 11 | 63.6 | 12.7 | 6.4 | 3.2 |
| 12 | 69.3 | 13.9 | 6.9 | 3.5 |
| 13 | 75.1 | 15.0 | 7.5 | 3.8 |
| 14 | 80.9 | 16.2 | 8.1 | 4.0 |
| 15 | 86.7 | 17.3 | 8.7 | 4.3 |
| 16 | 92.4 | 18.5 | 9.2 | 4.6 |
| 17 | 98.2 | 19.6 | 9.8 | 4.9 |
| 18 | 104.0 | 20.8 | 10.4 | 5.2 |
| 19 | 109.8 | 22.0 | 11.0 | 5.5 |
| 20 | 115.8 | 23.1 | 11.6 | 5.8 |
| 21 | 121.3 | 24.3 | 12.1 | 6.1 |
| 22 | 127.1 | 25.4 | 12.7 | 6.4 |
| 23 | 132.9 | 26.6 | 13.3 | 6.6 |
| 24 | 138.7 | 27.7 | 13.9 | 6.9 |
| 25 | 144.5 | 28.9 | 14.4 | 7.2 |

In some examples, ozone can be added to water to generate aqueous ozone. For example, venturi valves can be used to insert tiny bubbles of ozone into a pressurized flow of water. As another example, an aqueous ozone reactor can be used to add ozone to a static tank of water which can be used on-demand. Aqueous ozone has several applications. For example, aqueous ozone can be sprayed on surfaces to sanitize the surfaces, which would be attractive for Clean In Place (CIP). Additionally or alternatively, aqueous ozone can be added to clean water to wash or cool food (e.g., fruits, vegetables, meats, and the like), removing pathogens on the food surface. Additionally or alternatively, aqueous ozone can be used to clean contaminated wash water, for example, waste water from washing foods. This can be referred to as water reconditioning. After the water is reconditioned, the ozone can be removed from the water before reusing.

The cleaning device(s) 304 can include a UVC light controller 312 that controls the UVC light unit(s) 314. UVC light unit(s) 314 can emit UVC light. UVC-based disinfection is achieved by shining UVC light on a surface for a sufficiently long time. Ultraviolet light is a band of the electromagnetic spectrum, occupying the space between visible violet and x-rays. The wavelengths between 200 and 280 nm can be readily absorbed by pathogens, resulting in destruction of the pathogens, called UV germicidal irradiation (UVGI). The maximum absorption by DNA and RNA occurs at a wavelength of 264 nm. In some examples, a wavelength of 254 nm can be used because it can be produced by low-pressure mercury-vapor lamps.

The cumulative amount of energy delivered by germicidal UVC light over the exposure time is referred to as fluence. The period during which energy delivered by UVC light lands on a given unit area is referred to as the exposure time. The rate at which energy delivered by UVC light lands on a given unit area is referred to as the irradiance (W/m²). In some examples, light meters can be used to measure irradiance. For example, light meters can be placed in the enclosed space of the monitored location, or around items/objects to be cleaned/disinfected/sanitized. Values mascaraed by the light meters can be used to determine the exposure time. Formula 6 shows the relationship between the exposure time (in seconds), the fluence ((J/m²), and the irradiance (W/m²).

$$E x p o s u r e T i m e \left(s\right) = \frac{F l u e n c e \left(J / {m}^{2}\right)}{I r r a d i a n c e \left(W / {m}^{2}\right)} \qquad \text{Formula 6}$$

The irradiance is a function of the light unit’s intensity, the distance from the light unit to the target, and the angle of incidence. For example, a single 25 W 254 nm germicidal UVC compact fluorescent lamp (CFL) bulb may deliver an irradiance of about 10 W/m² at a distance of 20 cm. That means to achieve a fluence of, for example, 400 J/m², an exposure time of 40 s is needed. Such a short exposure time raises high expectations for the effectiveness of UVC. However, the irradiance drops rapidly as distance and incident angle increase, and there may be shadows. Irradiance degrades rapidly with distance in accordance with the inverse square law. This means that objects that are slightly further away from the UVC light units receive much less irradiance or disinfecting energy. Moreover, irradiance degrades as the angle of incidence increases. The irradiance is greatest when the light shines perpendicular to the plane. If the intensity of the UVC light unit increases, then the distant surfaces may get more energy, but objects close to the UVC light unit may be at risk of over-exposure, which could affect certain materials. Alternatively, the exposure time can be increased. Since UVC light is less ineffective over large distances, multiple UVC units may be used, or the UVC light units may be configured to move robotically to reposition.

Dosage values of UVC light are fluence values of UVC light. Dosage values of UVC light may vary based on different types of pathogens. In some examples, a dosimeter can be used to measure fluence, i.e. the total exposure dosage that has been applied. For example, dosimeter cards can change color when the exposure dosage has crossed various dosage thresholds. The dosimeter cards are like the radioactivity badges worn by people working near nuclear power.

The cleaning device(s) 304 can include a plurality of sensors that detects internal and/or external parameters associated with monitored locations. The plurality of sensors may include ozone concentration sensor(s) 316 configured to detect ozone concentrations associated with monitored locations, temperature sensor(s) 318 configured to detect internal and/or external temperatures associated with monitored locations, pressure sensor(s) 320 configured to detect internal and/or external pressures associated with monitored locations, humidity sensor(s) 322 configured to detect internal and/or external humidity associated with monitored locations, ATP sensor(s) 324 configured to detect ATP levels associated with monitored locations, and other sensor (s) 326. In some examples, since ATP is a molecular compound found in all living tissue and forms the basis of energy creation, the ATP level may indicate the presence of any organic material in Relative Light Units (RLUs). However, it should be noted that though microorganisms like bacteria and fungus contain ATP, so does non-pathogenic material. There, measuring ATP is a technique for indirectly measuring sanitation efficacy. ATP sensors may deliver instant results regarding ATP levels, but ATP sensors do not measure the pathogenic load directly. In some examples, if an accurate level of pathogenic load is needed, samples may be collected from the monitored location to be grown and counted colonies in Petri dishes.

The cleaning device(s) 304 can include other sensor(s) based on actual needs, such as image sensors, sound sensors, action sensors, light sensors, photoelectric sensors, ultrasonic sensors, particle sensors, smoke sensors, fire sensors, lidar sensors, time of flight sensors, radar sensors, and so on. This disclosure is not limited thereto.

The cleaning device(s) 304 can include destructor(s) 328. There is a safe continuous exposure limit for humans to gaseous ozone, usually under 0.1 ppm. Extended exposure to ozone with concentrations between 0.1 ppm and 1 ppm can cause respiratory complications. Exposure to ozone with a concentration of 50 ppm for 30 min may result in hazardous consequences for humans. Therefore, destructor(s) 328 can be used to make an ozone-rich environment safe for a human to re-enter. The air inside the monitored location may be pumped through the destructor(s) 328, which can convert the ozone back to harmless oxygen. In some examples, the destructor(s) 328 may decompose the ozone into oxygen by pumping ozonated air through a chamber filled with catalytic pellets. The catalyst is consumable and may be replaced periodically (e.g., every 6 months or other period of time) to keep the destructor(s) 328 working well.

The cleaning device(s) 304 can include a humidifier(s) 330. For the ozone to work well, humidity may be provided to the enclosed space of the monitored location. In some examples, the humidifier(s) 330 can increase humidity (moisture) in the enclosed space of the monitored location by injecting atomized water into the enclosed space. The humidifier(s) 330 can include different type of humidifiers such as evaporative humidifiers, impeller humidifiers, ultrasonic humidifiers, and the like. For example, an evaporative humidifier may include a reservoir, a wick, and a fan. The wick can be made of a porous material that absorbs water from the reservoir and provides a larger surface area for water to evaporate from. The fan may be adjacent to the wick and blows air onto the wet wick to aid in the evaporation of the water. As another example, an impeller humidifier (a type of cool mist humidifier) may use a rotating disc to fling water at a diffuser, which breaks the water into fine droplets that float into the air. As yet another example, an ultrasonic humidifier may use a ceramic diaphragm vibrating at an ultrasonic frequency to create water droplets that silently exit the humidifier in the form of cool fog. The ultrasonic humidifier may use a piezoelectric transducer to create a high frequency mechanical oscillation in a film of water. This forms an extremely fine mist of droplets about one micron in diameter, that is quickly evaporated into the air flow. In some examples, the humidifier(s) 330 can be portable or installed inside the monitored location. In some examples, the cleaning system 300 can control the output of ozone, UVC, and humidity. In some examples, the humidifier(s) 330 can include a humidifier and a dehumidifier to increase or otherwise decrease a humidity of air in an environment according to one or more control parameters.

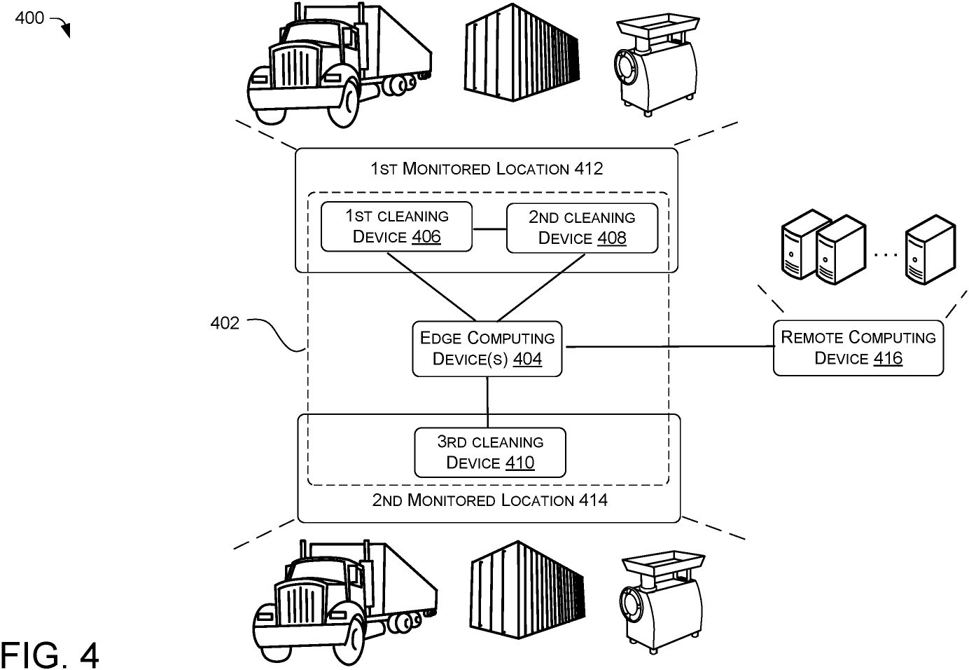

FIG. 4 illustrates an example environment 400 where a cleaning system 402 includes cleaning devices deployed at different monitored locations in accordance with examples of the disclosure. The cleaning system 402 includes a plurality of devices such as edge computing device(s) 404 configured to gather and process data described herein, a first cleaning device 406, a second cleaning device 408, and a third cleaning device 410 configured to perform cleaning/disinfection/sanitization. The first cleaning device 406, the second cleaning device 408, and the third cleaning device 410 may be placed at the monitored locations within an enclosed space to be cleaned/disinfected/sanitized. Additionally alternatively, each of the first cleaning device 406, the second cleaning device 408, and the third cleaning device 410 may include an enclosed space to contain the items/objects to be cleaned/disinfected/sanitized.

As an example, the first cleaning device 406 and the second cleaning device 408 may be deployed at a first monitored location 412, and the third cleaning device 410 may be deployed at a second monitored location 414. It should be understood that though FIG. 4 shows two monitored locations and three cleaning devices, there may be other numbers of monitored locations and cleaning devices. In various examples, the edge computing device(s) 404 can correspond to the edge computing device(s) 102 of FIG. 1 and the edge computing device(s) 202 of FIG. 2. The first, second, and third cleaning devices 406, 408, and 410 can correspond to the cleaning device(s) 108 of FIG. 1 and the cleaning device(s) 304 of FIG. 3. In various examples, the edge computing device(s) 404, the first cleaning device 406, the second cleaning device 408, and the third cleaning device 410 can communicate wired or wirelessly.

Each of the first monitored location 412 and the second monitored location 414 may include, but is not limited to, transportation instruments (e.g., school buses, trucks, shipping containers, and the like), farm settings (e.g., incubators, hatchery rooms, and the like), medical settings (e.g., operation rooms, waiting rooms, healthcare clinic rooms, ambulances, and the like), public locations (e.g., cinemas, restaurants, offices, stores, hotels, clubhouses, and the like), home settings (e.g., dining rooms, kitchens, appliances, bedrooms, and the like), and the like. Additionally or alternatively, Each of the first monitored location 412 and the second monitored location 414 may include things or items/objects to be cleaned/disinfected/sanitized such as cargos (e.g., goods carried in trucks, vans, airplanes, trains, ships, and the like), food processing equipment (e.g., meat grinders, cutting boards, utensils, and the like), food (e.g., fruit, vegetables, chilling poultry, and the like), surgical tools (e.g., scissors, surgical blades, knives and scalpels, and the like), furniture (e.g., tables, chairs, sofas, carpets, curtains, and the like), decorations (e.g., paintings, plants, ornaments, and the like), and the like.

The edge computing device(s) 404 may communicate with the remote computing device(s) 416. In some examples, the edge computing device(s) 404 may send data to the remote computing device(s) 416. In some examples, the data may indicate the internal temperature of the first monitored location 412/second monitored location 414, the internal pressure of the first monitored location 412/second monitored location 414, internal humidity of the first monitored location 412/second monitored location 414, dimension of the first monitored location 412/second monitored location 414, the external temperature of the first monitored location 412/second monitored location 414, the external pressure of the first monitored location 412/second monitored location 414, external humidity of the first monitored location 412/second monitored location 414, or materials to be disinfected. In some examples, the data may include the weather conditions (temperature, wind, daytime, nighttime, rain, etc.) associated with the first monitored location 412/the second monitored location 414, or the like. In some examples, data may include the timestamp associated with the data.

The remote computing device(s) 406 may send control parameters to the edge computing device(s) 404 regarding how to control the cleaning devices 406, 408, and 410. In some examples, the control parameters may include an ozone concentration level to pulse up to, an ozone concentration level to let the ozone concentration to fall to, an order of emitting UVC or ozone at a monitored location, a length of time of emitting the UVC, a humidity level to control a humidifier unit, an order of increasing humidity (e.g., a humidity profile over time), a maximum or minimum humidity level for a cleaning environment, an expected humidity level, an ozone concentration level for setting for a particular monitored location, an expected ozone decay rate for a particular monitored location when clean, and the like.

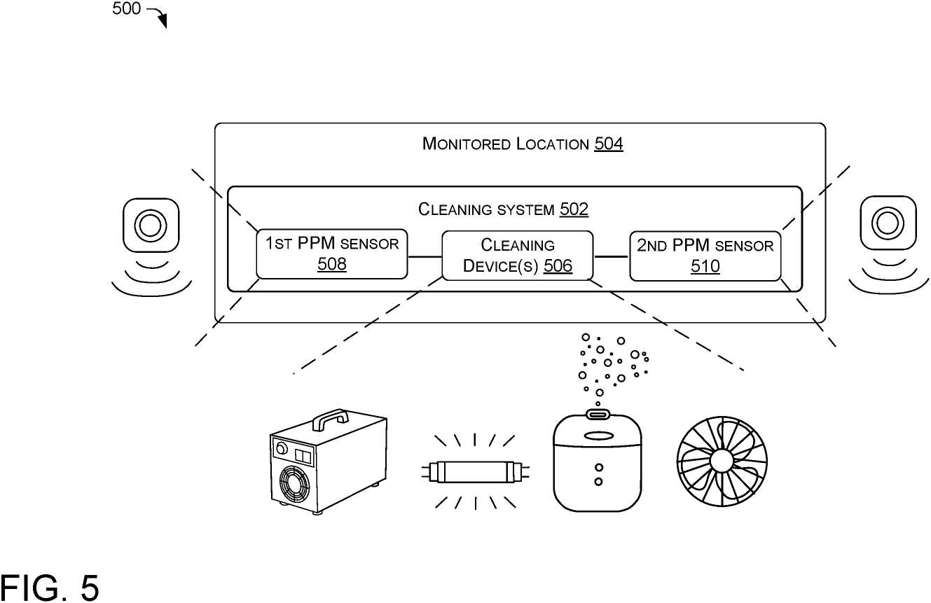

FIG. 5 illustrates an example environment 500 where a cleaning system 502 is placed inside a monitored location 504 in accordance with examples of the disclosure. In various examples, the monitored location 504 can correspond to the monitored location(s) 104 of FIG. 1, the first and the second monitored locations 412 and 414 of FIG. 4. The cleaning system 502 includes a plurality of devices such as cleaning device(s) 506 configured to perform cleaning/disinfection/sanitization of the monitored location 504, a first PPM sensor 508, and a second PPM sensor 510. Though FIG. 5 shows two PPM sensors, there may be any number or type of sensors.

The cleaning device(s) 506 may include ozone generator(s), UVC light units, and/or humidifier(s). In some examples, the ozone generator(s), UVC light units, and the humidifier(s) can work individually, in combination, or alternately to perform cleaning/disinfection/sanitation. In some examples, the cleaning device(s) 506 may be implemented in different forms. For example, an immobile monitored location like a room can have a permanent installation of ozone generators, UVC light units, and the humidifier(s), while a mobile monitored location like a transportation vehicle may use a portable ozone generator, UVC light units, and the humidifiers.

Additionally, the cleaning device(s) 506 may be laid out to achieve optimal/desired results. For example, buses and trains are long and narrow, which presents some challenges. As an example, when an area in a bus is under exposure, the UVC light units may be intensified or new UVC light sources may be added so that each area of the bus could be sufficiently exposed. On the other hand, when an area is over-exposed, materials may be damaged, and thus the UVC light units may be dimmed or reduced. The layout of UVC light units may be configured to achieve a balanced exposure.

The cleaning device(s) 506 may include air circulation devices (s) such as fans, ventilators, and the like. Ozone (2.14 kg/m³) is denser than oxygen (1.43 kg/m³) and air (1.29 kg/m³) and tends to sink if mixed with air. If the diffusion of ozone is poor in the monitored location, it is easy to get different concentrations throughout the monitored location. For example, within a refrigerator-sized enclosed space, the disparity may be over 10 ppm. In some examples, forced-air circulation may be used to facilitate the diffusion of the ozone. Additionally, fast moving air may also increase the reactivity of the ozone, and thus facilitate the cleaning/disinfection/sanitization. Additionally, fast moving air may cause the ozone to decompose into oxygen quickly.

The cleaning system 502 may include a plurality of PPM sensors (i.e., the first PPM sensor 508 and the second PPM sensor 510) deployed throughout the monitored location 504 to detect ozone concentrations throughout the monitored location 504. In some examples, the plurality of PPM sensors (i.e., the first PPM sensor 508 and the second PPM sensor 510) may be installed throughout the monitored location 504. In some examples, the plurality of PPM sensors (i.e., the first PPM sensor 508 and the second PPM sensor 510) may be portable or movable robotically (e.g., based on control parameters).

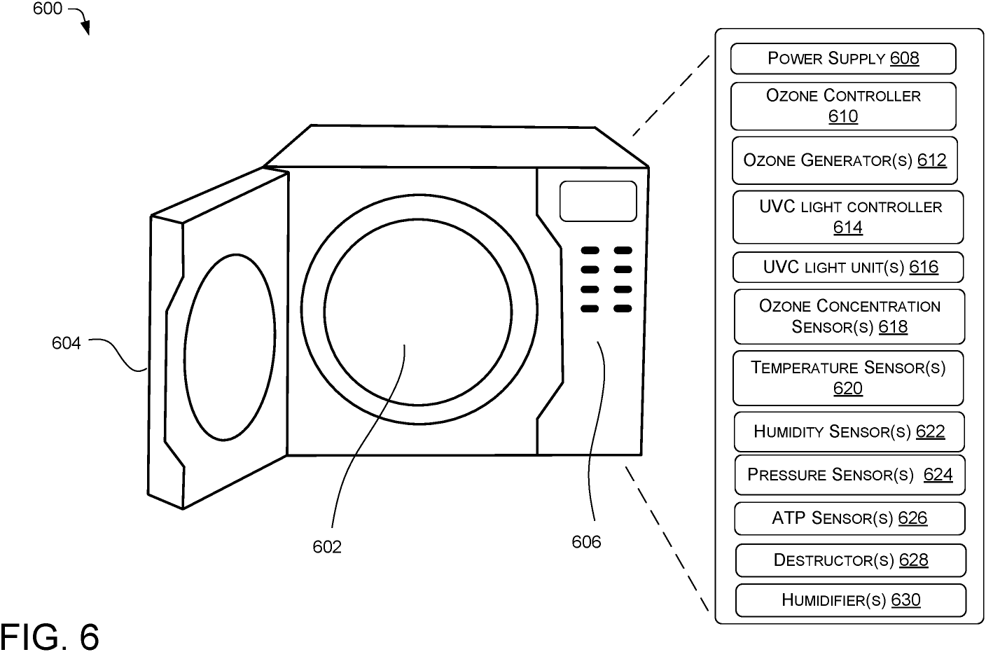

FIG. 6 illustrates an example cleaning system 600 with an internal chamber 602 to clean/disinfect/sanitize items/objects in accordance with examples of the disclosure. In some examples, the cleaning system 600 may have a custom size. In some examples, the cleaning system 600 may be constructed from ozone-resistant materials, such as stainless-steel, and the like. In some examples, the cleaning system 600 may be implemented as a non-thermal autoclave, that is, an enclosure that achieves a level of disinfection without the use of heat or steam. The non-thermal autoclave is helpful because many items/objects that need to be disinfected are not heat or moisture resistant.

The cleaning system 60 may have a lockable door 604. The load/items/objects to be cleaned/disinfected/sanitized may be placed inside chamber 602 and door 604 may be shut. An interactive panel 606 may be used to display information and/or receive input from the user. The door 604 may lock and cannot be reopened while the cleaning/disinfection/sanitization is in progress.

The cleaning system 600 can include a power supply 608 that receives power from a network such as a power grid. In some examples, the power supply 608 can include protection units to protect the cleaning system 600 from surge current, overvoltage, overcurrent, leakage, and the like. In some examples, in the event of long-term power loss, the cleaning system 600 may halt with the door 604 remaining locked. When power resumes, the cleaning/disinfection/sanitization cycle may restart from the beginning.

The cleaning system 600 can include an ozone controller 610 that controls the ozone generator(s) 612. The ozone generator(s) 612 can generate ozone from air or oxygen. In some examples, the ozone generator(s) 612 may take the ambient air, converting the oxygen content into ozone. In some examples, an air pump (not shown) may pass ambient air through a dehumidifier to improve the ozonation efficiency. The ozone generator(s) 612 may take the dried air, converting the oxygen content into ozone. The ozone generator(s) 612 may inject/deliver the ozone into the chamber 602. Ozone concentration sensor(s) 618 may track the ozone concentrations within the chamber 602 so that ozone generator(s) 612 can pause and resume the ozonation with micro-doses to maintain the ozone concentration inside the chamber. Additional details are given throughout this disclosure.

The cleaning system 600 can include a UVC light controller 614 that controls the UVC light unit(s) 616. The UVC light unit(s) 616 can emit UVC light. UVC light unit(s) 616 can improve the disinfection efficiency, as well as add a significant effect on stubborn pathogens such as spores. In some examples, ozone generator(s) 612 and UVC light unit(s) 616 can work individually, in combination, or alternately to achieve cleaning/disinfection/sanitization results.

The cleaning system 600 can include a plurality of sensors that detects internal and/or external parameters associated with cleaning system 600. The plurality of sensors may include ozone concentration sensor(s) 618 configured to detect ozone concentrations inside the chamber 602, temperature sensor(s) 620 configured to detect internal and/or external temperatures, humidity sensor(s) 622 configured to detect internal and/or external humidity, pressure sensor(s) 624 configured to detect internal and/or external pressures, ATP sensor(s) 626 configured to detect ATP levels. Additionally, the cleaning system 600 may use AIoT sensors (e.g., PPM sensors, temperature sensors, pressure sensors, humidity sensors, and the like) to take environmental readings so the cleaning system 600 can estimate the time for cleaning/disinfection/sanitization.

The panel 606 may display status information including, but is not limited to, internal and/or external temperatures, internal and/or external humidity, internal and/or external pressures, ATP levels, an estimate for the completing the cleaning/disinfection/sanitization. When the certain conditions are met, for example, the decay rate of ozone inside the chamber 602 meets an expected ozone decay rate, the air inside the chamber 602 may be pumped through destructor(s) 628, which can convert the ozone back to oxygen. In some examples, the destructor(s) 628 may decompose the ozone into oxygen by pumping ozonated air through a chamber filled with catalytic pellets. The catalyst is consumable and may be replaced periodically (e.g., every 6 months, or other regular or irregular interval) to keep the destructor working well.

The cleaning system 600 can include humidifier(s) 630. In some examples, the humidifier(s) 630 can increases humidity (moisture) in the chamber 602 by injecting atomized water into the chamber 602. The humidifier(s) 630 can include different type of humidifiers such as evaporative humidifiers, impeller humidifiers, ultrasonic humidifiers, and the like. In some examples, the humidifier(s) 630 can be portable or as a part of the cleaning system 600. In some examples, the cleaning system 600 can control the output of ozone, UVC, and humidity.

Once the cleaning system 600 confirms a safe level of ozone, the door 604 unlocks, and the cleaning/disinfection/sanitization process is complete. In some examples, the cleaning system 600 may communicate with the remote computing device(s) (not shown) to upload data regarding the anonymized settings used and sensor readings to a computing device.

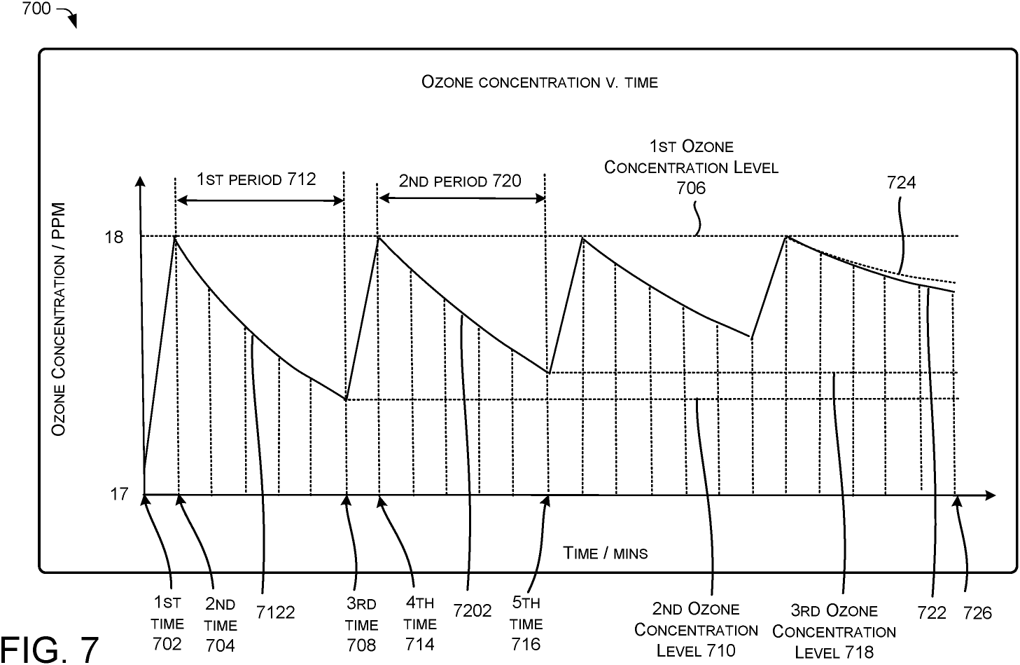

FIG. 7 illustrates an example graph 700 of ozone concentration (in PPM) versus time (in minutes) in accordance with examples of the disclosure. Referring to FIG. 4, the vertical axis represents the ozone concentration (in PPM), while the horizontal axis represents time in minutes. In some examples, the ozone concentrations can be detected by the ozone concentration sensors such as the ozone concentration sensor(s) 218 of FIG. 2, the ozone concentration sensor(s) 316 of FIG. 3, the first and second PPM sensors 508, and 510 of FIG. 5, the ozone concentration sensor(s) 618 of FIG. 6, and the like. In some examples, the ozone concentrations can be detected in an enclosed space at monitored locations such as the monitored location(s) 104 of FIG. 1, the first and second monitored locations 412 and 414 of FIG. 4, the monitored location 504 of FIG. 5, the chamber 602 of FIG. 6, and the like.

A pathogen-free room, chamber, or autoclave, will still react with any ozone injected into it, converting it back into oxygen over time. This is a baseline, or “clean signature,” which reflects how a sanitized environment behaves. But the same environment, with the addition of some pathogens, will consume the ozone faster, and that is an “unclean signature.” Over the span of a few minutes, as the room becomes increasingly decontaminated, and with each log reduction in pathogens, the signatures will get closer and closer until they match.

Referring to FIG. 7, at a first time 702, an ozone generator starts to pulse up ozone in an enclosed space of the monitored location, and the ozone concentration starts to increase. At a second time 704, the ozone concentration reaches a first ozone concentration level 706 (e.g., 18 ppm), and the ozone generator stops pulsing the ozone. Between the second time 704 and a third time 708, the ozone concentration decreases. At the third time 708, the ozone concentration decreases to a second concentration level 710. During a first period 712 between the second time 704 and the third time 708, the ozone concentration has a first decay curve 7122. The first decay curve 7122 may have a first decay constant.

At the third time 708, the ozone generator starts to pulse up ozone in the enclosed space of the monitored location. At a fourth time 714, the ozone concentration reaches the first ozone concentration level 706 (e.g., 18 ppm) again, and the ozone generator stops pulsing the ozone. Between the fourth time 714, and a fifth time 716, the ozone concentration decreases. At the fifth time 716, the ozone concentration decreases to a third ozone concentration level 718. During a second period 720 between the fourth time 714 and the fifth time 716, the ozone concentration has a second decay curve 7202. The second decay curve 7202 may have a second decay constant.

The decay curve of ozone can be defined by Formula 7.

$$c = k ∗ {e}^{m t} \qquad \text{Formula 7}$$

In Formula 7, t represents time in seconds; c represents ozone concentration in ppm at time t; mt represents instantaneous exponential decay constant at time t; k represents ozone offset level constant (causes the curve to translate vertically but does not affect the shape).

The rate of ozone injection may be linear. For example, the same number of ozone molecules are injected into the enclosed space of the monitored location, during the first 10 mins as the next 10 mins. However, because the decay of ozone begins immediately and is more pronounced with higher ozone concentrations, the ozone concentration increases linearly at low ozone concentrations, but is influenced by the decay of ozone at high ozone concentrations. It gets harder and harder to maintain high ozone concentrations because ozone collapses faster as the ozone concentration goes up.

Over time, and in a static environment such as an enclosed room or a chamber, the concentration of gaseous ozone decays at an exponential rate that is governed by two factors - the presence of reactants and the half-life of ozone. The contaminants in the environment, both inorganic and organic, will react with some of the ozone. Another factor is the natural decay of ozone based on its half-life, which is also influenced by temperature, humidity, and air flow among others.

The half-life of a chemical is the time taken for half the chemical molecules to disappear, that is, to convert into something else, for example, ozone to oxygen. The half-life of gaseous ozone can vary from minutes to a few days, depending on at least the following: temperature (the higher the temperature, the shorter the half-life of ozone), humidity (the higher the humidity, the shorter the half-life of ozone), air flow rate (the higher the air flow rate, the shorter the half-life of ozone), air-based reactants (the higher the concentration of the air-based reactants, the shorter the half-life of ozone), or surface-based reactants (the higher the concentration of the surface-based reactants, the shorter the half-life of ozone). For example, if the ozone concentration drops from 2 ppm at 1 min to 1 ppm at 9 min, the half-life of ozone is 8 mins.

The rate of half-life destruction of ozone is exponential. As an example, the ozone concentration drops by 50% during the first 10 mins, and then a further 50% during the next 10 mins. For example, if the initial ozone concentration is 16 ppm, after 10 mins, the ozone concentration is 8 ppm, and after another 10 mins, the ozone concentration is 4 ppm.

The instantaneous exponential decay of ozone concentration may be used as a leading indicator of environmental disinfection. In practice, after the environment has been fully disinfected once, the decay profile of the “maximally disinfected room” can be extracted. When the environment is increasingly disinfected, the decay curves of ozone concentration become shallower. For example, the second decay curve 7202 is shallower than the first decay curve 7122. When ozone is injected into a contaminated environment, the exponential decay constant will be more negative. After each top-up pulse of ozone (in the process of maintaining a specified concentration level), the exponential decay constant will gradually increase positively until it approaches the threshold defined as maximally disinfected. In some examples, when the exponential decay of ozone concentration in a static environment approaches the curve for the same but fully disinfected environment, the contaminants, including pathogens, can be assumed or otherwise determined to have been eliminated. For example, the shallowest decay curve 722 may indicate that the enclosed space of the monitored location can be deemed as cleaned/disinfected/sanitized.

The graph 700 reflects the cleaning/disinfection/sanitization process that uses feedback from ozone concentrations sensors and edge computing devices to determine when a target concentration level has been attained and then pulses the ozone generator for a brief period allowing the ozone to decay. The ever-changing decay gradient is in effect a real-time measure of the chemical demand for oxidation, which is an indicator of the level of disinfection.

Following a pulse of ozone, the environment gets cleaner with each passing second, so the decay constant becomes an instantaneous value, that varies over time, up to the next pulse, and then continues to change. In some examples, the instantaneous decay constant may be continually recalculated based on the prior three ozone concentration readings, although any number of readings can be used.

The industry uses the exposure concept of Concentration-Time (CT) to specify how much, and how long something should be applied. Doses of ozone are represented by CT values. Referring to Formula 8, a CT value is a product of the concentration and time.

$$C T = c ∗ t \qquad \text{Formula 8}$$

In Formula 8, CT is the CT value; c is the residual concentration in ppm; t is the time in minutes.

For example, 15 ppm for 5 min would be a CT value of 75 ppm-min. The concentration of ozone can be reduced (within reason) as time elapses, and vice-versa. As a point of reference, an ozone concentration of 20 ppm is fairly high, and some meters cannot read above such a level.

Formula 8 is accurate when the residual concentration does not vary during the time interval, or if the residual concentration varies linearly and a mean value can be used. For example. if the CT is 16 ppm-min, the dosage could equivalently use 2 ppm for 8 mins, 4 ppm for 4 mins, or 16 ppm for 1 min.

More generally, referring to Formula 9, the CT is defined by the area under the curve. This is particularly relevant when a single initial dose of ozone is applied, and the residual concentration declines over a longer time.

$$C T \text{=} {\int }_{{t}_{i n i t i a l}}^{{t}_{f i n a l}} k * {e}^{m t} d t \qquad \text{Formula 9}$$

In Formula 9, CT is the CT value; t represents time in seconds; tᵢₙᵢₜᵢₐₗ represents the initial time; t_{final} represents the final time; c represents ozone concentration in ppm at time t; mt represents instantaneous exponential decay constant at time t; k represents ozone offset level constant.

However, with frequent readings from one or more sensors, the concentration decline over a short time period can be approximated to be linear, and so the mean value for each time period can be used. The CT value for n measurements taken d mins apart, where each value c is taken at time t, is given by Formula 10.

$$C T = {\sum }_{n = 1}^{n - 1} \frac{{}_{}^{c}{\left(n - 1\right)} {d}^{+ c} n d}{2} \qquad \text{Formula 10}$$

As shown in FIG. 7, after each pulse of ozone, in an effort to maintain an ozone concentration around the first ozone concentration level 706 (e.g., 18 ppm), the decay progressively levels off. Approaching the expected decay curve 724 with exponential decay constant m means that the enclosed space of the monitored location becomes a more disinfected environment. The area (from first time 702 to the sixth time 726) under the curve (extended to y=0) is the total CT value applied.

In some examples, a “current” decay curve can be compared to one or more “previous” decay curves. For example, the decay curve associated with the period 720 can be compared to the decay curve associated with the period 712. In some examples, if the difference between the decay curves is less than a threshold, the cleaning environment or object to be cleaned can be considered to be clean, sanitized, and/or disinfected. In some examples, the difference can be based on the slope of the decay curve, a time period to go from a first ozone concentration to a second ozone concentration, and the like. Additional metrics and algorithms for evaluating a level of cleanliness is discussed herein.

There may be practical limitations in the cleaning/disinfection/sanitization process. For example, an excessively dilute ozone concentration could be completely ineffective, and excessively high ozone concentrations could damage delicate commodities, such as soft fruit. Additionally, if the cleaning/disinfection/sanitization time is too long, it may not be compatible with a short window of opportunity for overnight disinfection of food processing parts such as grinder blades, because plants cannot be down for too long.

Usually, CT values may include an expected log reduction. For example, a CT value is 75 ppm-min for a 2 log reduction, and no assumption should be made that increasing the CT value will result in a 3 log reduction. In some instances, the CT value for the greatest log reduction can be detected. In practice, ozone may max out at a 1-3 log reduction in Salmonella, and boosting the cleaning effect with UVC may be helpful. However, in some instances, higher log reductions may occur. The estimated CT values to inactivate pathogens may vary based on the types of pathogens. For example, an ozone concentration of 5 ppm for 58 seconds (or an ozone concentration of 10 ppm for 30 seconds) may be needed to inactivate Campylobacter jejuni. The effectiveness, or log reduction, depends on the types of pathogens present, the types of material the biofilm is on, the tolerance to oxidation by the commodity, and the time available. For example, E. coli is far easier to destroy than the spore-forming Clostridium. The surfaces of raspberries are infinitely less hardy than stainless steel grinding blades. The ozone concentration and UVC intensity levels required for a one-hour cycle may be different from a three-hour cycle.

In some examples, with each completed run of the cleaning/disinfection/sanitization process, the cleaning system may record data such as the CT, peak concentration, mean concentration, elapsed time, temperature, humidity, load type, and so on. With the user’s permission (e.g., based on a user setting), the cleaning system may export the data to the remote computing device(s). In some examples, the data may be stored in a cloud-based, pooled but anonymized database. The data may be used to feed a machine learned model. The remote computing device(s) may continuously refine the algorithms, which may lead to greater log reductions with lower ozone concentrations and in shorter times. The focus may also be on discovering CT values for different scenarios, constrained by the max concentration level that can be tolerated by a commodity, yet sufficient to destroy the pathogens.

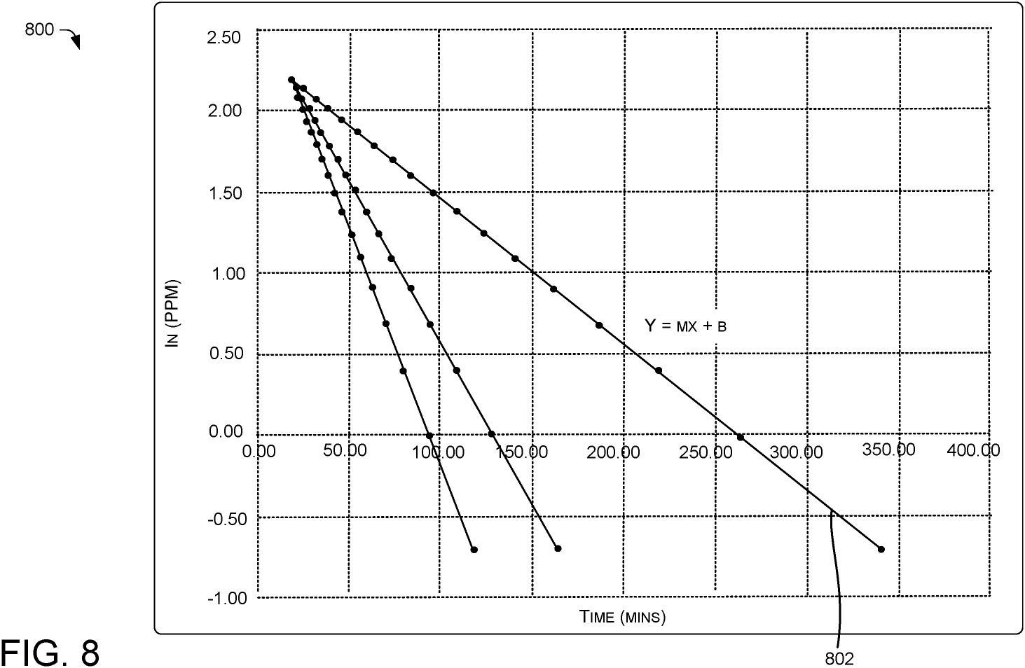

FIG. 8 illustrates an example graph 800 which converts the exponential ozone concentration curves into straight lines.

In some examples, the ozone concentration can be plotted on a logarithmic concentration scale, and thus the trendline would be linear, which is easier to use for predictions and analysis. As described herein, the decay curve of ozone is defined by Formula 7. Referring to Formula 11, taking the natural log of both sides of Formula 7 produces the linear equation with slope m.

$$c = k ∗ {e}^{m t} \qquad \text{Formula 7}$$

$$\text{ln} \left(c\right) = m t \text{+ ln} \left(k\right) \qquad \text{Formula 11}$$

In Formula 11, t represents time in seconds; c represents ozone concentration in ppm at time t; mt represents instantaneous exponential decay constant at time t; k represents ozone offset level constant (causes the curve to translate vertically but does not affect the shape).

For example, the slope of the cleanest line 802 can be referred to as the variable m.

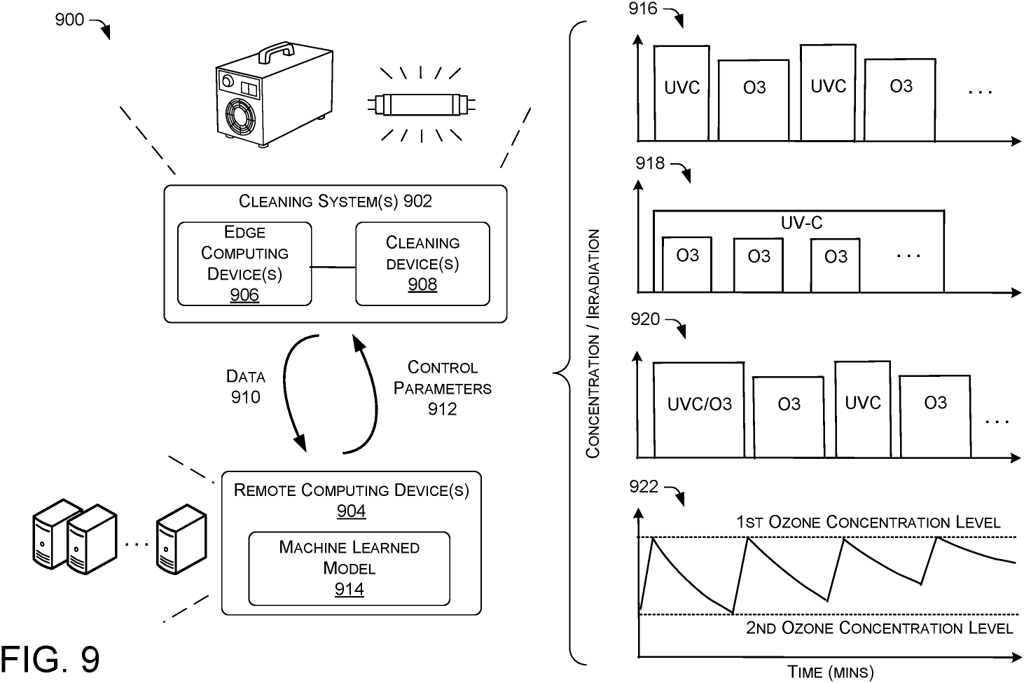

FIG. 9 illustrates an example environment 900 including one or more cleaning systems 902 and one or more remote computing devices 904 in accordance with examples of the disclosure. In various examples, the cleaning system(s) 902 can correspond to the cleaning system(s) 102 of FIG. 1, the cleaning system(s) 300 of FIG. 3, the cleaning system(s) 402 of FIG. 4, the cleaning system(s) 502 of FIG. 5, and the cleaning system 600 of FIG. 6. In various examples, the remote computing device(s) 904 can correspond to the remote computing device(s) 110 of FIG. 1, the remote computing device(s) 204 of FIG. 2, and the remote computing device(s) 416 of FIG. 4. The cleaning system(s) 902 and the remote computing device(s) 904 can communicate wired or wirelessly.

The cleaning system(s) 902 may include edge computing device(s) 906 and cleaning device(s) 908. In various examples, the edge computing device(s) 906 the edge computing device(s) 102 of FIG. 1, the edge computing device(s) 202 of FIG. 2, the computing device(s) 302 of FIG. 3, and the edge computing device(s) 404 of FIG. 4. The cleaning device(s) 908 can correspond to the cleaning device(s) 108 of FIG. 1, the cleaning device(s) 304 of FIG. 3, the first, second, and third cleaning devices 406, 408, and 410 of FIG. 4, the cleaning device(s) 506 of FIG. 5, and so on. In some examples, the cleaning device(s) 908 may perform cleaning/disinfection/sanitization utilizing ozone and UVC, controlled by the edge computing device(s) 906.

The edge computing device(s) 906 may communicate with the remote computing device(s) 904. In some examples, the edge computing device(s) 906 may send data 910 to the remote computing device(s) 904. In some examples, the data may indicate internal temperature, internal pressure, internal humidity, dimension of the enclosed space of the monitored location, external temperature, external pressure, external humidity, materials to be disinfected, and the like. In some examples, the data may include the weather conditions (temperature, wind, daytime, nighttime, rain, etc.), or the like. In some examples, data may include the timestamp associated with the data.

The remote computing device(s) 904 may send control parameters 912 to the edge computing device(s) 906 of the cleaning system(s) 902. In some examples, the remote computing device(s) 904 may include a machine learned model 914. In some examples, the machine learned model 914 can be configured and/or trained to determine how to refine the cleaning/disinfection/sanitization process. In some examples, the machine learned model 914 can be configured and/or trained to determine control parameters, which may be sent to the edge computing device(s) 906.

In some examples, the machine learned model 914 can correspond to the machine learned model 250, as discussed above.

The control parameters 912 may include an ozone concentration level to pulse up to, an ozone concentration level to let the ozone concentration to fall to, an order of emitting UVC or ozone at a monitored location, a length of time of emitting the UVC, a humidity level to control a humidifier unit, an order of increasing humidity (e.g., a humidity profile over time), a maximum or minimum humidity level for a cleaning environment, an expected humidity level, an ozone concentration level for setting for a particular monitored location, an expected ozone decay rate for a particular monitored location when clean, and the like. In some examples, control parameters 912 may include various schemes to control the ozone generation, UVC irradiation, and/or a humidifier unit. In some examples, as shown in graph 916, the ozone generation and the UVC irradiation can be performed alternately. In some examples, as shown in graph 918, the UVC irradiation can be performed continuously while the ozone generation can be performed with pulses. In some examples, as shown in graph 920, the UVC irradiation and the ozone generation can be performed at the same time and then alternately. In some examples, as shown in graph 922, the ozone generation can be pulsed to reach a first ozone concentration level multiple times until the ozone decay rate matches an expected ozone decay rate for a particular monitored location when clean.

In some examples, the machine learned model 914 can perturb various control parameters, send the control parameters to one or more cleaning systems 902, and receive log data detailing the resulting cleaning operation. Accordingly, the machine learned model 914 can continuously updated the model to determine optimal control strategies based on improved cleaning performance.

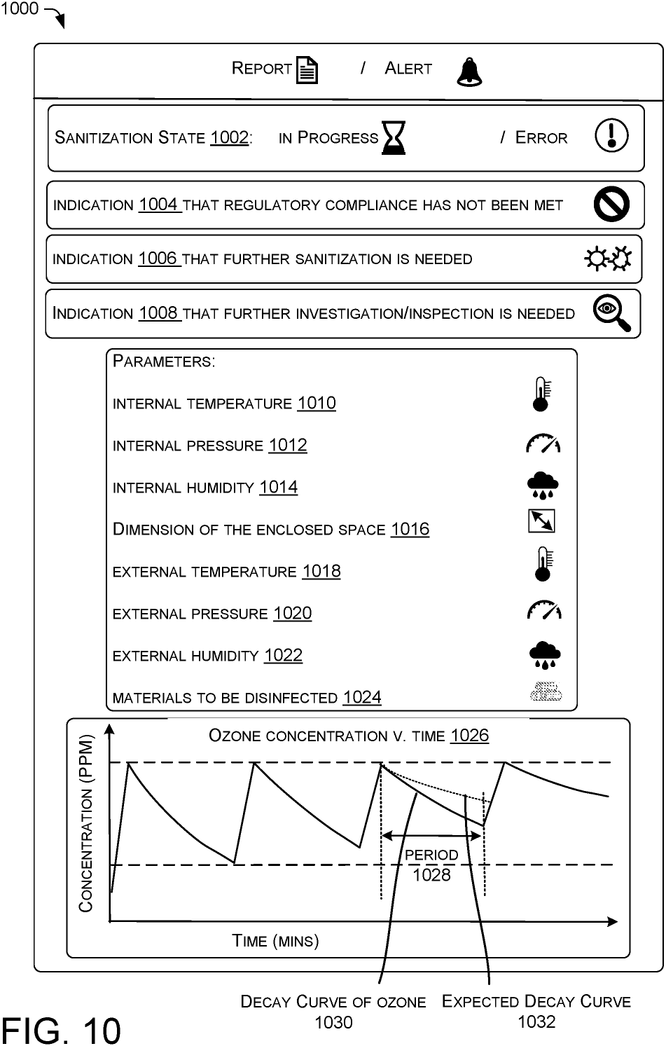

FIG. 10 illustrates a graphical user interface (GUI) 1000 illustrating a report/alert page for a monitored location in accordance with examples of the disclosure. In various examples, the monitored location can correspond to the monitored location(s) 104 of FIG. 1, the first and the second monitored locations 412 and 414 of FIG. 4, and the monitored location 504 of FIG. 5.

The GUI 1000 can be displayed via user device(s) such as the user device(s) 112 of FIG. 1, the user device(s) 206 of FIG. 2, and the like. As can be understood in the context of this disclosure, reports and/or alerts can be conveyed in any manner, including but not limited to a text message, web-portal, email, website, push notification, pull notification, specific applications (“apps”), or the like. In some examples, the GUI 1000 may illustrate the sanitization state associated with the monitored location, one or more indications, parameters associated with the monitored location, graphs associated with the monitored location, and the like.

For example, the GUI 1000 illustrates that a sanitization state 1002 is an in progress state and/or an error state. The GUI 1000 also illustrates an indication 1004 that regulatory compliance has not been met, an indication 1006 that further sanitization is needed, and an indication 1008 that further investigation/inspection is needed. Though FIG. 10 shows three indications (1004, 1006, and 1008), there may be other indications. The GUI 1000 also illustrates multiple parameters including internal temperature 1010 of the monitored location, internal pressure 1012 of the monitored location, internal humidity 1014 of the monitored location, dimension of the enclosed space 1016 of the monitored location, external temperature 1018 of the monitored location, external pressure 1020 of the monitored location, external humidity 1022 of the monitored location, and materials to be disinfected 1024. It should be understood that the GUI 1000 may include other parameters, and this disclosure is not limited thereto.

The GUI 1000 may also illustrate one or more graphs reflecting the cleaning/disinfection/sanitization process of the monitored location. For example, the GUI 1000 shows an ozone concentration versus time graph 1026. Graph 1026 may also show whether the decay curves of ozone match expected decay curves under similar conditions. For example, in a period 1028, there is a disparity between the decay curve of ozone 1030 and the expected decay curve 1032. For example, the GUI 1000 discussed herein can be provided in an alert to notify the disparity.

As can be understood in the context of this disclosure, any date and/or time period can be selected via the GUI 1000, causing the GUI 1000 to dynamically update the information displayed. In this manner, the GUI 1000 may provide a personalized user interface illustrating information specified by and relevant to a user. In some examples, the indications 1004, 1006, and 1008 can be associated with a color or other visual indicator to provide high-level impression. For example, the white (or uncolored) of an indication can indicate normal state, while a red color of an indication can indicate an emergent state. Thus, the GUI 1000 provides a simple overview of the cleaning/disinfection/sanitization process of the monitored location that allows a user to have a deep understanding of the profile of the monitored location.

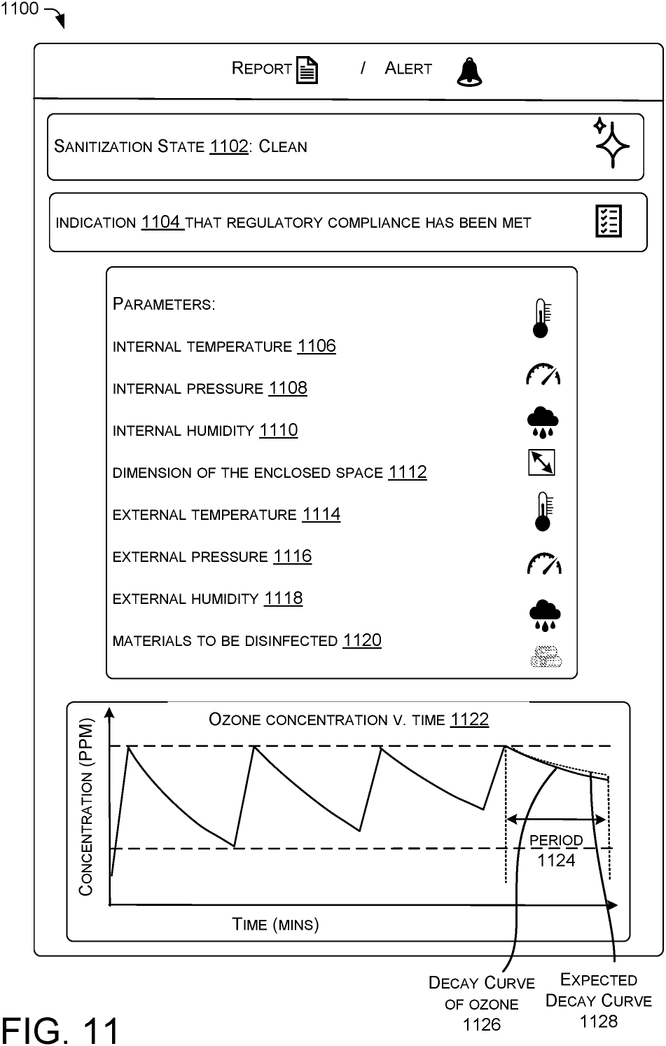

FIG. 11 illustrates a graphical user interface (GUI) 1100 illustrating a report/alert page for a monitored location in accordance with examples of the disclosure. In various examples, the monitored location can correspond to the monitored location(s) 104 of FIG. 1, the first and the second monitored locations 412 and 414 of FIG. 4, and the monitored location 504 of FIG. 5.

The GUI 1100 can be displayed via user device(s) such as the user device(s) 112 of FIG. 1, the user device(s) 206 of FIG. 2, and the like. As can be understood in the context of this disclosure, reports and/or alerts can be conveyed in any manner, including but not limited to a text message, web-portal, email, website, push notification, pull notification, specific applications (“apps”), or the like. In some examples, the GUI 1100 may illustrate the sanitization state associated with the monitored location, one or more indications, parameters associated with the monitored location, graphs associated with the monitored location, and the like.

For example, the GUI 1100 illustrates that a sanitization state 1102 is a clean state. The GUI 1100 also illustrates an indication 1104 that regulatory compliance has been met. Though FIG. 11 shows one indication 1104, there may be other indications. The GUI 1100 also illustrates multiple parameters including internal temperature 1106 of the monitored location, internal pressure 1108 of the monitored location, internal humidity 1110 of the monitored location, dimension of the enclosed space 1112 of the monitored location, external temperature 1114 of the monitored location, external pressure 1116 of the monitored location, external humidity 1118 of the monitored location, and materials to be disinfected 1120. It should be understood that the GUI 1100 may include other parameters, and this disclosure is not limited thereto.

The GUI 1100 may also illustrate one or more graphs reflecting the cleaning/disinfection/sanitization process of the monitored location. For example, the GUI 1100 shows an ozone concentration versus time graph 1122. Graph 1122 may also show whether the decay curves of ozone match expected decay curves under similar conditions. For example, in a period 1124, the decay curve of ozone 1126 matches the expected decay curve 1128. In some examples, when the decay curve of ozone concentration approaches the expected decay curve for the same but fully disinfected environment, all contaminants, including pathogens, can be assumed to have been eliminated.

As can be understood in the context of this disclosure, any date and/or time period can be selected via the GUI 1100, causing the GUI 1100 to dynamically update the information displayed. In this manner, the GUI 1100 may provide a personalized user interface illustrating information specified by and relevant to a user. In some examples, the indication 1104 can be associated with a color or other visual indicator to provide high-level impression. For example, the white (or uncolored) of an indication can indicate normal state, while a red color of an indication can indicate an emergent state. Thus, the GUI 1100 provides a simple overview of the cleaning/disinfection/sanitization process of the monitored location that allows a user to have a deep understanding of the profile of the monitored location.

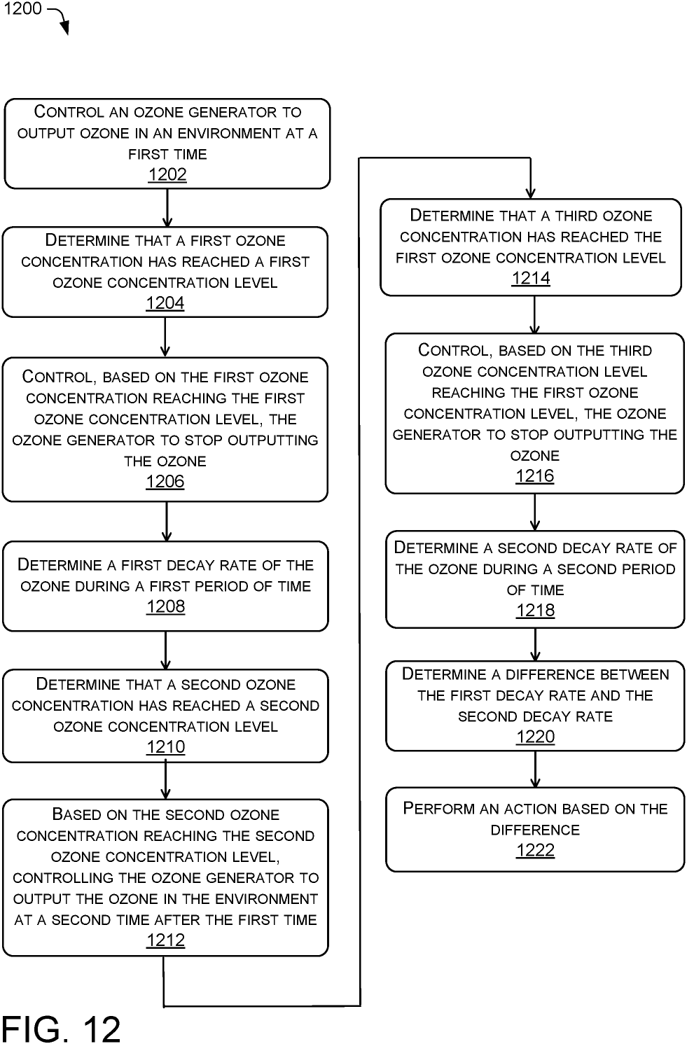

FIGS. 12-15 illustrate example processes in accordance with examples of the disclosure. These process are illustrated as a logical flow graph, each operation of which represents a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be omitted or combined in any order and/or in parallel to implement the processes.

FIG. 12 illustrates an example cleaning/disinfection/sanitization process 1200 in accordance with examples of the disclosure. In some examples, at least some operations of the process 1200 may be performed by the cleaning system(s), the remote computing device(s), and/or the user device(s) as described herein.

At 1202, operations may include controlling an ozone generator to output ozone in an environment at a first time. For example, at the first time, the ozone generator may start to pulse the ozone into the environment such that the ozone concentration in the environment may increase. In some examples, the environment is an enclosed environment in which at least one of: the environment is to be disinfected or the environment contains an object to be disinfected .

At 1204, operations may include determining that a first ozone concentration has reached a first ozone concentration level. In some examples, the first ozone concentration level is useful to achieve the disinfection results in the environment. In some examples, the first ozone concentration is measured by ozone concentration sensor(s).

At 1206, operations may include controlling, based on the first ozone concentration reaching the first ozone concentration level, the ozone generator to stop outputting the ozone.

At 1208, operations may include determining a first decay rate of the ozone during a first period of time. In some examples, the first decay rate of the ozone may be determined based on Formula 7 and Formula 11 as described herein.

At 1210, operations may include determining that a second ozone concentration has reached a second ozone concentration level. In some examples, the second ozone concentration is measured by ozone concentration sensor(s).

At 1212, operations may include based on the second ozone concentration reaching the second ozone concentration level, controlling the ozone generator to output the ozone in the environment at a second time after the first time.

At 1214, operations may include determining that a third ozone concentration has reached the first ozone concentration level.

At 1216, operations may include controlling, based on the third ozone concentration reaching the first ozone concentration level, the ozone generator to stop outputting the ozone.

At 1218, operations may include determining a second decay rate of the ozone during a second period of time. In some examples, the second period of time is after the first period of time. In some examples, the second decay rate of the ozone may be determined based on Formula 7 and Formula 11 as described herein.

At 1220, operations may include determining a difference between the first decay rate and the second decay rate. In some examples, the operations may further include monitoring differences in decay rates of the ozone in the environment over a plurality of periods of time.

At 1222, operations may include performing an action based on the difference. In some examples, the action includes determining, based at least in part on the difference meeting or exceeding a threshold, that the environment is not disinfected. In some examples, the action includes determining, based at least in part on the difference being below the threshold, that the environment is clean. In some examples, the operations further include sending a signal indicative of the difference to a remote computing device.

In some examples, the operations may further include measuring one or more of: the internal temperature of the environment, the internal pressure of the environment, internal humidity of the environment, environment size, external temperature, external pressure, external humidity, or materials to be disinfected.

In some examples, the operations may further include controlling an air circulation device to facilitate the ozone to diffuse in the environment. The air circulation device may be fans, ventilators, and the like. Since ozone is denser than oxygen and air, ozone tends to sink if mixed with air. If the diffusion of ozone is poor in the monitored location, it is easy to get different concentrations throughout the monitored location. Therefore, forced-air circulation may be used to facilitate the diffusion of the ozone. Additionally, fast moving air may also increase the reactivity of the ozone, and thus facilitate the cleaning/disinfection/sanitization. Additionally, fast moving air may cause the ozone to decompose into oxygen quickly.

In some examples, the ozone concentrations may be measured by multiple sensors placed at different places in the environment. For example, the multiple sensors may include a first sensor and a second sensor. The process 1200 may further include determining a sensor difference between a first reading of the first sensor and a second reading of the second sensor, and sending an indication of the sensor difference to a remote computing device. The difference between readings of sensors may indicate that the environment is not yet clean. The process 1200 may further include controlling UVC light unit(s) to irradiate UVC light based at least in part on the difference.

In some examples, the process 1200 may further include receiving a parameter from a remote computing device; and controlling the ozone generator based at least in part on the parameter. In some examples, the parameter includes one or more of the first concentration level, the second concentration level, or an instruction for operating the ozone generator relative to a UVC light.

In some examples, the process 1200 may further include monitoring environmental data for regulatory compliance.

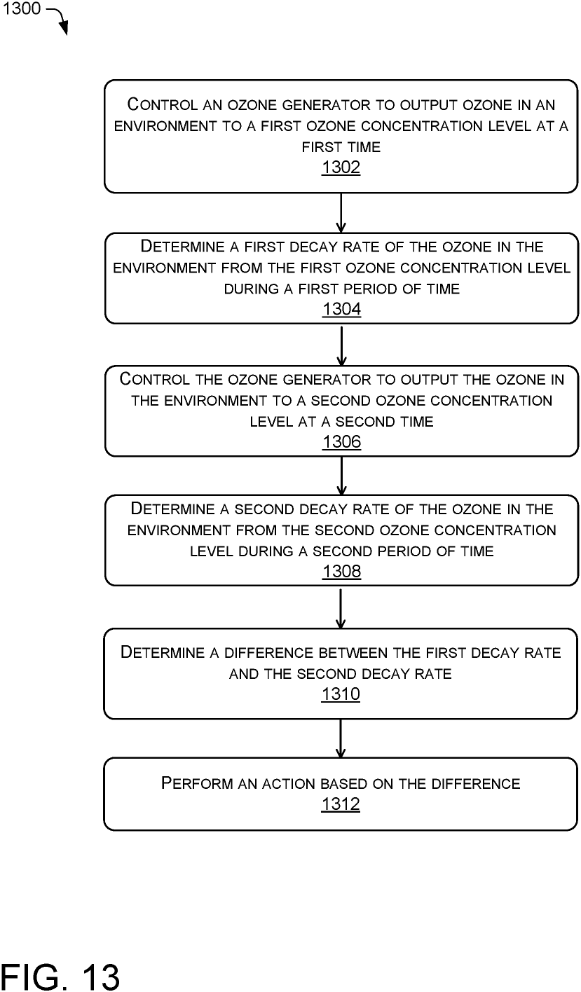

FIG. 13 illustrates an example cleaning/disinfection/sanitization process 1300 in accordance with examples of the disclosure. In some examples, at least some operations of the process 1300 may be performed by the cleaning system(s), the remote computing device(s), and/or the user device(s) as described herein.

At 1302, operations may include controlling an ozone generator to output ozone in an environment to a first ozone concentration level at a first time. For example, at the first time the ozone generator may start to pulse the ozone into the environment such that the ozone concentration in the environment may increase. In some examples, the environment is an enclosed environment in which at least one of: the environment is to be disinfected or the environment contains an object to be disinfected .

At 1304, operations may include determining a first decay rate of the ozone in the environment from the first ozone concentration level during a first period of time. In some examples, the first decay rate of the ozone may be determined based on Formula 7 and Formula 11 as described herein.

At 1306, operations may include controlling the ozone generator to output the ozone in the environment to a second ozone concentration level at a second time.

At 1308, operations may include determining a second decay rate of the ozone in the environment from the second ozone concentration level during a second period of time. In some examples, the second decay rate of the ozone may be determined based on Formula 7 and Formula 11 as described herein.

At 1310, operations may include determining a difference between the first decay rate and the second decay rate. In some examples, the operations may further include monitoring differences in decay rates of the ozone in the environment over a plurality of periods of time.

At 1312, operations may include performing an action based on the difference. In some examples, the action includes determining, based at least in part on the difference meeting or exceeding a threshold, that the environment is not disinfected. In some examples, the action includes determining, based at least in part on the difference being below the threshold, that the environment is clean. In some examples, the operations further include sending a signal indicative of the difference to a remote computing device.

In some examples, the operations may further include measuring one or more of: the internal temperature of the environment, the internal pressure of the environment, internal humidity of the environment, environment size, external temperature, external pressure, external humidity, or materials to be disinfected.

In some examples, the operations may further include controlling an air circulation device to facilitate the ozone to diffuse in the environment. The air circulation device may be fans, ventilators, and the like. Since ozone is denser than oxygen and air, ozone tends to sink if mixed with air. If the diffusion of ozone is poor in the monitored location, it is easy to get different concentrations throughout the monitored location. Therefore, forced-air circulation may be used to facilitate the diffusion of the ozone. Additionally, fast moving air may also increase the reactivity of the ozone, and thus facilitate the cleaning/disinfection/sanitization. Additionally, fast moving air may cause the ozone to decompose into oxygen quickly.

In some examples, the ozone concentrations may be measured by multiple sensors placed at different places in the environment. For example, the multiple sensors may include a first sensor and a second sensor. The process 1300 may further include determining a sensor difference between a first reading of the first sensor and a second reading of the second sensor, and sending an indication of the sensor difference to a remote computing device. The difference between readings of sensors may indicate that the environment is not yet clean. The process 1300 may further include controlling UVC light unit(s) to irradiate UVC light based at least in part on the difference.

In some examples, the process 1300 may further include receiving a parameter from a remote computing device; and controlling the ozone generator based at least in part on the parameter. In some examples, the parameter includes one or more of the first concentration level, the second concentration level, or an instruction for operating the ozone generator relative to a UVC light.

In some examples, the process 1300 may further include monitoring environmental data for regulatory compliance.

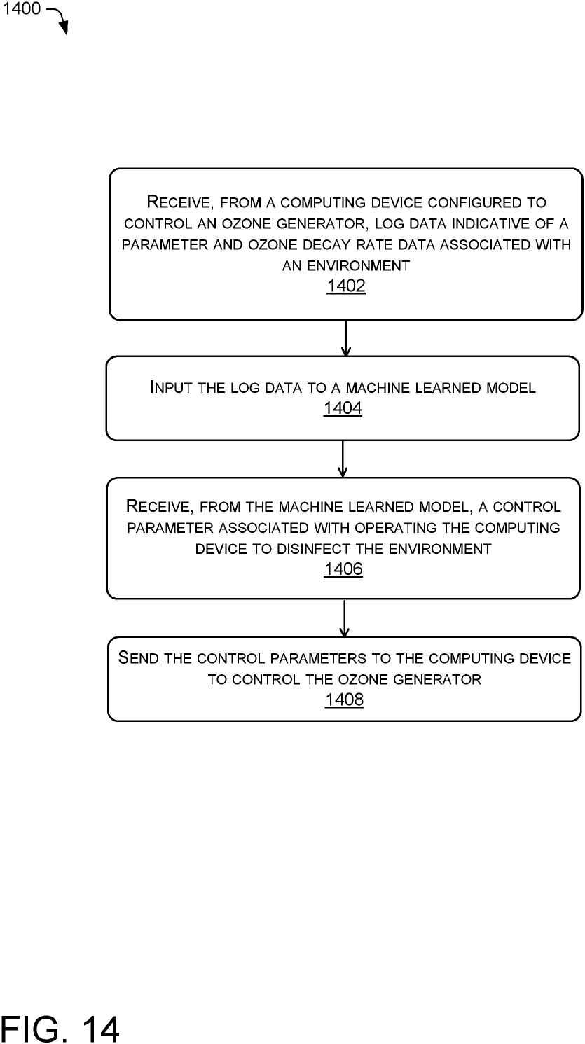

FIG. 14 illustrates an example cleaning/disinfection/sanitization process 1400 in accordance with examples of the disclosure. In some examples, at least some operations of the process 1400 may be performed by the cleaning system(s), the remote computing device(s), and/or the user device(s) as described herein.

At 1402, operations may include receiving, from a computing device configured to control an ozone generator, log data indicative of a parameter, and ozone decay rate data associated with an environment. In some examples, the parameter includes one or more of internal temperature of the environment, the internal pressure of the environment, internal humidity of the environment, environment size, external temperature, external pressure, external humidity, or materials to be disinfected. In some examples, the ozone decay rate data is indicative of a rate of decay of the ozone in the environment after the computing device controls the ozone generator to output to a desired concentration level. In some examples, the operations may further include receiving, from the computing device, data indicative of ATP measurement or a pathogen measurement; and associating the data indicative of the ATP measurement or the pathogen measurement with the log data. In some examples, the operations may further include receiving log data from a plurality of computing devices in a plurality of environments.

At 1404, operations may include inputting the log data to a machine learned model. In some examples, the machine learned model can include any models, algorithms, and/or machine learning algorithms, as discussed herein.

At 1406, operations may include receiving, from the machine learned model, a control parameter associated with operating the computing device to disinfect the environment. In some examples, the control parameter controls at least one of an ozone concentration level to pulse up to, an ozone concentration level to let the ozone concentration to fall to, an order of emitting UVC or ozone in an environment, a length of time of emitting UVC, a humidity level to control a humidifier unit, an order of increasing humidity (e.g., a humidity profile over time), a maximum or minimum humidity level for a cleaning environment, an expected humidity level, an ozone concentration level for setting for a particular environment, or an expected ozone decay rate for a particular environment when clean.

At 1408, operations may include sending the control parameters to the computing device to control the ozone generator (or another component, such as the UVC light, a humidifier, and the like).

In some examples, the ozone concentrations may be measured by multiple sensors placed at different places in the environment. For example, the multiple sensors may include a first sensor and a second sensor. The process 1400 may further include receiving a sensor difference between a first reading of the first sensor and a second reading of the second sensor. The difference between readings of sensors may indicate that the environment is not yet clean.

In some examples, the process 1400 may further include receiving, from the computing device, environmental data for regulatory compliance.

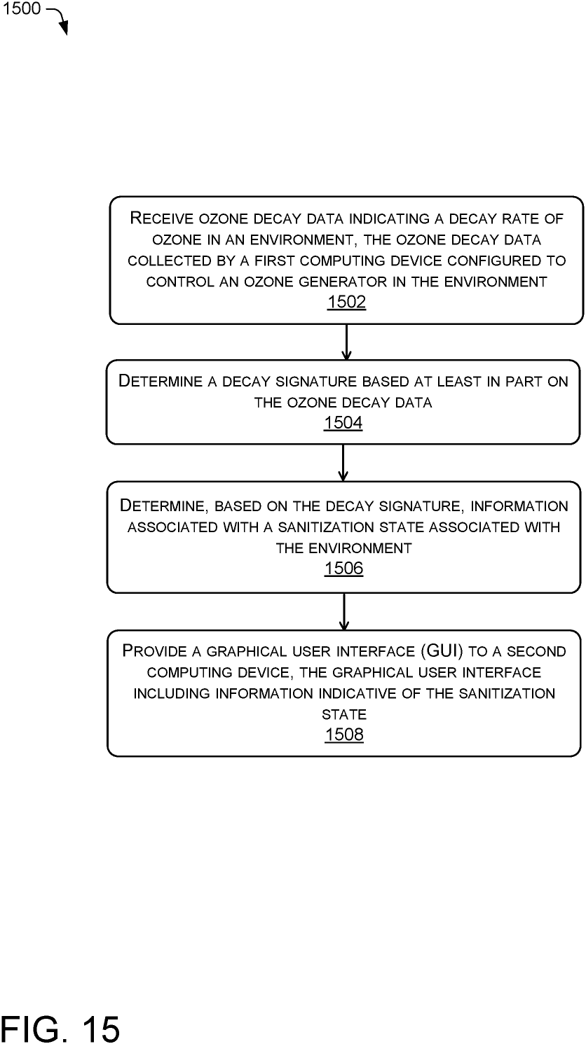

FIG. 15 illustrates an example cleaning/disinfection/sanitization process 1500 in accordance with examples of the disclosure. In some examples, at least some operations of the process 1500 may be performed by the cleaning system(s), the remote computing device(s), and/or the user device(s) as described herein.

At 1502, operations may include receiving ozone decay data indicating a decay rate of ozone in an environment, the ozone decay data collected by a first computing device configured to control an ozone generator in the environment.

At 1504, operations may include determining a decay signature based at least in part on the ozone decay data.

At 1506, operations may include determining, based on the decay signature, information associated with a sanitization state associated with the environment.

At 1508, operations may include providing a graphical user interface (GUI) to a second computing device, the graphical user interface including information indicative of the sanitization state. In some examples, the sanitization state may include at least one of: a clean state, an in-progress state, or an error state. In some examples, the GUI may include an indication to further sanitize the environment or an object in the environment, an indication that further sanitization is needed, an indication regarding whether regulatory compliance has been met, and so on. In some examples, the GUI may further include information regarding at least one of internal temperature of the environment, the internal pressure of the environment, internal humidity of the environment, environment size, external temperature, external pressure, external humidity, or materials to be disinfected. In some examples, the GUI may further include information regarding decay rates of the ozone in the environment over a plurality of periods of time. In some examples, the operations may further include comparing the decay signature to historical decay data to determine the sanitization state. In some examples, the GUI can correspond to one or more GUIs illustrated in FIGS. 10 and 11.

In some examples, the process 1500 may further include inputting the decay signature into a machine learned model; and receiving, from the machine learned model, the sanitization state.

In some examples, the process 1500 may further include determining whether a disinfection cycle of the environment is longer than a cycle threshold; and upon determining that the disinfection cycle of the environment is longer than the cycle threshold, sending, to the second computing device, the GUI including an indication that an inspection over the environment is needed.

### EXAMPLE CLAUSES

A: A system comprising: one or more processors; and one or more non-transitory computer-readable media storing instructions executable by the one or more processors, wherein the instructions, when executed, cause the system to perform operations comprising: controlling an ozone generator to output ozone in an environment at a first time; determining that a first ozone concentration has reached a first ozone concentration level; controlling, based on the first ozone concentration reaching the first ozone concentration level, the ozone generator to stop outputting the ozone; determining a first decay rate of the ozone during a first period of time; determining that a second ozone concentration has reached a second ozone concentration level; based on the second ozone concentration reaching the second ozone concentration level, controlling the ozone generator to output the ozone in the environment at a second time after the first time; determining that a third ozone concentration has reached the first ozone concentration level; controlling, based on the third ozone concentration reaching the first ozone concentration level, the ozone generator to stop outputting the ozone; determining a second decay rate of the ozone during a second period of time; determining a difference between the first decay rate and the second decay rate; and performing an action based on the difference.

B: The system of paragraph A, wherein the action comprises: determining, based at least in part on the difference meeting or exceeding a threshold, that the environment is not disinfected; or determining, based at least in part on the difference being below the threshold, that the environment is clean.

C: The system of paragraph A, the operations further comprising sending a signal indicative of the difference to a remote computing device.

D: The system of paragraph A, wherein the second period of time is after the first period of time.

E: The system of paragraph A, the operations further comprising measuring one or more of: internal temperature of the environment, internal pressure of the environment, internal humidity of the environment, environment size, external temperature, external pressure, external humidity, or materials to be disinfected.

F: A method comprising: controlling an ozone generator to output ozone in an environment to a first ozone concentration level at a first time; determining a first decay rate of the ozone in the environment from the first ozone concentration level during a first period of time; controlling the ozone generator to output the ozone in the environment to a second ozone concentration level at a second time; determining a second decay rate of the ozone in the environment from the second ozone concentration level during a second period of time; determining a difference between the first decay rate and the second decay rate; and performing an action based on the difference.

G: The method of paragraph F, further comprising controlling an air circulation device to facilitate the ozone to diffuse in the environment.

H: The method of paragraph F, further comprising measuring the first ozone concentration level and the second ozone concentration level from multiple sensors in the environment.

I: The method of paragraph H, wherein the multiple sensors include a first sensor and a second sensor, the method further comprising: determining a sensor difference between a first reading of the first sensor and a second reading of the second sensor; and sending an indication of the sensor difference to a remote computing device.

J: The method of paragraph F, further comprising controlling at least one of an ultraviolet C (UVC) light unit or a humidifier unit based at least in part on the difference.

K: The method of paragraph F, further comprising: receiving a parameter from a remote computing device; and controlling the ozone generator based at least in part on the parameter, wherein the parameter includes one or more of the first ozone concentration level, the second ozone concentration level, or an instruction for operating the ozone generator relative to a UVC light.

L: The method of paragraph F, further comprising monitoring differences in decay rates of the ozone in the environment over a plurality of periods of time.

M: The method of paragraph F, further comprising monitoring environmental data for regulatory compliance.

N: The method of paragraph F, wherein the environment is an enclosed environment in which at least one of: the environment is to be disinfected or the environment contains an object to be disinfected.

O: One or more non-transitory computer-readable media storing instructions that, when executed, cause one or more processors to perform operations comprising: controlling an ozone generator to output ozone in an environment to a first ozone concentration level at a first time; determining a first decay rate of the ozone in the environment from the first ozone concentration level during a first period of time; controlling the ozone generator to output the ozone in the environment to a second ozone concentration level at a second time; determining a second decay rate of the ozone in the environment from the second ozone concentration level during a second period of time; determining a difference between the first decay rate and the second decay rate; and performing an action based on the difference.

P: The one or more non-transitory computer-readable media of paragraph O, wherein the action comprises: determining, based at least in part on the difference meeting or exceeding a threshold, that the environment is not disinfected; or determining, based at least in part on the difference being below the threshold, that the environment is clean.

Q: The one or more non-transitory computer-readable media of paragraph O, further comprising sending a signal indicative of the difference to a remote computing device.

R: The one or more non-transitory computer-readable media of paragraph O, wherein the second period of time is after the first period of time.

S: The one or more non-transitory computer-readable media of paragraph O, the operations further comprising measuring one or more of: internal temperature of the environment, internal pressure of the environment, internal humidity of the environment, environment size, external temperature, external pressure, external humidity, or materials to be disinfected.

T: The one or more non-transitory computer-readable media of paragraph O, the operations further comprising controlling a fan to circulate the ozone in the environment.

U: A system comprising: one or more processors; and one or more non-transitory computer-readable media storing instructions executable by the one or more processors, wherein the instructions, when executed, cause the system to perform operations comprising: receiving, from a computing device configured to control an ozone generator, log data indicative of a parameter and ozone decay rate data associated with an environment; inputting the log data to a machine learned model; receiving, from the machine learned model, a control parameter associated with operating the computing device to disinfect the environment; and sending the control parameters to the computing device to control the ozone generator.

V: The system of paragraph U, wherein the parameter includes one or more of: internal temperature of the environment, internal pressure of the environment, internal humidity of the environment, environment size, external temperature, external pressure, external humidity, or materials to be disinfected.

W: The system of paragraph U or V, wherein the ozone decay rate data is indicative of a rate of decay of the ozone in the environment after the computing device controls the ozone generator to output to a desired concentration level.

X: The system of any of paragraphs U-W, wherein the control parameter controls at least one of: an ozone concentration level to pulse up to; an ozone concentration level to let the ozone concentration to fall to; an order of emitting ultraviolet C (UVC) or ozone in an environment; a length of time of emitting UVC; a humidity level to control a humidifier unit; an expected humidity level; an ozone concentration level for setting for a particular environment; or an expected ozone decay rate for a particular environment when clean.

Y: The system of any of paragraphs U-X, the operations further comprising receiving a plurality of log data from a plurality of computing devices in a plurality of environments, wherein the machine learned model is based at least in part on the plurality of log data.

Z: A method comprising: receiving, from a computing device configured to control an ozone generator, log data indicative of a parameter and ozone decay rate data associated with an environment; inputting the log data to a machine learned model; receiving, from the machine learned model, a control parameter associated with operating the computing device to disinfect the environment; and sending the control parameter to the computing device to control the ozone generator.

AA: The method of paragraph Z, wherein the parameter includes one or more of: internal temperature of the environment, internal pressure of the environment, internal humidity of the environment, environment size, external temperature, external pressure, external humidity, or materials to be disinfected.

AB: The method of paragraph AA, wherein the ozone decay rate data is indicative of a rate of decay of the ozone in the environment after the computing device controls the ozone generator to output to a desired concentration level.

AC: The method of paragraph AB, wherein the control parameter controls at least one of: an ozone concentration level to pulse up to; an ozone concentration to let the ozone concentration level to fall to; an order of emitting ultraviolet C (UVC) or ozone in a particular environment; a length of time of emitting UVC; a humidity level to control a humidifier unit; an expected humidity level; an ozone concentration level for setting for the particular environment; or an expected ozone decay rate for the particular environment when clean.

AD: The method of any of paragraphs Z-AC, further comprising receiving log data from a plurality of computing devices in a plurality of environments.

AE: The method of any of paragraphs Z-AD, wherein the machine learned model is a convolutional neural network.

AF: The method of any of paragraphs Z-AE, further comprising: receiving, from the computing device, data indicative of an adenosine triphosphate (ATP) measurement or a pathogen measurement; and associating the data indicative of the ATP measurement or the pathogen measurement with the log data.

AG: The method of any of paragraphs Z-AF, further comprising receiving an indication of a sensor difference between a first ozone concentration level measured by a first sensor in the environment and a second ozone concentration level measured by a second sensor in the environment.

AH: The method of any of paragraphs Z-AG, further comprising receiving, from the computing device, data for regulatory compliance, wherein the data comprises at least one of environmental pollution regulations, safety regulations, hygiene standards, or local regulations associated with a particular country or district.

AI: One or more non-transitory computer-readable media storing instructions that, when executed, cause one or more processors to perform operations comprising: receiving, from a computing device configured to control an ozone generator, log data indicative of a parameter and ozone decay rate data associated with an environment; inputting the log data to a machine learned model; receiving, from the machine learned model, a control parameter associated with operating the computing device to disinfect the environment; and sending the control parameters to the computing device to control the ozone generator.

AJ: The one or more non-transitory computer-readable media of paragraph AI, wherein the parameter includes one or more of: internal temperature of the environment, internal pressure of the environment, internal humidity of the environment, environment size, external temperature, external pressure, external humidity, or materials to be disinfected.

AK: The one or more non-transitory computer-readable media of paragraph AI or AJ, wherein the ozone decay rate data is indicative of a rate of decay of the ozone in the environment after the computing device controls the ozone generator to output to a desired concentration level.

AL: The one or more non-transitory computer-readable media of paragraph AK, wherein the control parameters control at least one of: an ozone concentration level to pulse up to; an ozone concentration level to let the ozone concentration to fall to; an order of emitting ultraviolet C (UVC) or ozone in an environment; a length of time of emitting UVC; a humidity level to control a humidifier unit; an expected humidity level; an ozone concentration level for setting for a particular environment; or an expected ozone decay rate for a particular environment when clean.

AM: The one or more non-transitory computer-readable media of any of paragraphs AI-AL, the operations further comprising receiving log data from a plurality of devices in a plurality of environments.

AN: The one or more non-transitory computer-readable media of paragraph AI, wherein the machine learned model is a convolutional neural network.

AO: A system comprising: one or more processors; and one or more non-transitory computer-readable media storing instructions executable by the one or more processors, wherein the instructions, when executed, cause the system to perform operations comprising: receiving ozone decay data indicating a decay rate of ozone in an environment, the ozone decay data collected by a first computing device configured to control an ozone generator in the environment; determining a decay signature based at least in part on the ozone decay data; determining, based on the decay signature, information associated with a sanitization state associated with the environment; and providing a graphical user interface (GUI) to a second computing device, the graphical user interface including information indicative of the sanitization state.

AP: The system of paragraph AO, wherein the sanitization state includes at least one of: a clean state, an in-progress state, or an error state.

AQ: The system of paragraph AO or AP, wherein the GUI includes an indication to further sanitize the environment or an object in the environment.

AR: The system of any of paragraphs AO-AQ, the operations further comprising comparing the decay signature to historical decay data to determine the sanitization state.

AS: The system of any of paragraphs AO-AR, the operations further comprising: inputting the decay signature into a machine learned model; and receiving, from the machine learned model, the sanitization state.

AT: A method comprising: receiving ozone decay data indicating a decay rate of ozone in an environment, the ozone decay data collected by a first computing device configured to control an ozone generator in the environment; determining a decay signature based at least in part on the ozone decay data; determining, based on the decay signature, information associated with a sanitization state associated with the environment; and providing a graphical user interface (GUI) to a second computing device, the GUI displaying information indicative of the sanitization state.

AU: The method of paragraph AT, wherein the sanitization state includes at least one of: a clean state, an in-progress state, or an error state.

AV: The method of paragraph AT or AU, wherein the GUI includes an instruction to further sanitize the environment or object in the environment.

AW: The method of any of paragraphs AT-AV, further comprising comparing the decay signature to historical decay data to determine the sanitization state.

AX: The method of any of paragraphs AT-AW, further comprising: inputting the decay signature into a machine learned model; and receiving, from the machine learned model, the sanitization state.

AY: The method of any of paragraphs AT-AX, wherein the GUI includes an indication regarding whether regulatory compliance has been met.

AZ: The method of any of paragraphs AT-AY, further comprising: determining whether a disinfection cycle of the environment is longer than a cycle threshold; and upon determining that the disinfection cycle of the environment is longer than the cycle threshold, sending, to the second computing device, the GUI including an indication that an inspection over the environment is needed.

BA: The method of any of paragraphs AT-AZ, the GUI further includes information regarding at least one of: internal temperature of the environment, internal pressure of the environment, internal humidity of the environment, environment size, external temperature, external pressure, external humidity, or materials to be disinfected.

BB: The method of any of paragraphs AT-BA, wherein the GUI further includes information regarding decay rates of the ozone in the environment over a plurality of periods of time.

BC: One or more non-transitory computer-readable media storing instructions that, when executed, cause one or more processors to perform operations comprising: receiving ozone decay data indicating a decay rate of ozone in an environment, the ozone decay data collected by a first computing device configured to control an ozone generator in the environment; determining a decay signature based at least in part on the ozone decay data; determining, based on the decay signature, information associated with a sanitization state associated with the environment; and providing a graphical user interface (GUI) to a second computing device, the graphical user interface including information indicative of the sanitization state.

BD: The one or more non-transitory computer-readable media of paragraph BC, wherein the sanitization state includes at least one of: a clean state, an in-progress state, or an error state.

BE: The one or more non-transitory computer-readable media of paragraph BC or BD, wherein the GUI includes an instruction to further sanitize the environment or an object in the environment.

BF: The one or more non-transitory computer-readable media of paragraph BE, the operations further comprising comparing the decay signature to historical decay data to determine the sanitization state.

BG: The one or more non-transitory computer-readable media of any of paragraphs BC-BF, the operations further comprising: inputting the decay signature into a machine learned model; and receiving, from the machine learned model, the sanitization state.

BH: The one or more non-transitory computer-readable media of any of paragraphs BC-BG, the GUI includes an indication regarding whether regulatory compliance has been met.

While the example clauses described above are described with respect to one particular implementation, it should be understood that, in the context of this document, the content of the example clauses can also be implemented via a method, device, system, and/or a computer-readable medium.

### CONCLUSION

While one or more examples of the techniques described herein have been described, various alterations, additions, permutations and equivalents thereof are included within the scope of the techniques described herein.

In the description of examples, reference is made to the accompanying drawings that form a part hereof, which show by way of illustration specific examples of the claimed subject matter. It is to be understood that other examples can be used and that changes or alterations, such as structural changes, can be made. Such examples, changes or alterations are not necessarily departures from the scope with respect to the intended claimed subject matter. While the steps herein can be presented in a certain order, in some cases the ordering can be changed so that certain inputs are provided at different times or in a different order without changing the function of the systems and methods described. The disclosed procedures could also be executed in different orders. Additionally, various computations that are herein need not be performed in the order disclosed, and other examples using alternative orderings of the computations could be readily implemented. In addition to being reordered, the computations could also be decomposed into sub-computations with the same results.

What is claimed is:

1. A system comprising:
one or more processors; and
one or more non-transitory computer-readable media storing instructions executable by the one or more processors, wherein the instructions, when executed, cause the system to perform operations comprising:
controlling an ozone generator to output ozone in an environment at a first time;
determining that a first ozone concentration has reached a first ozone concentration level;
controlling, based on the first ozone concentration reaching the first ozone concentration level, the ozone generator to stop outputting the ozone;
determining a first decay rate of the ozone during a first period of time;
determining that a second ozone concentration has reached a second ozone concentration level;
based on the second ozone concentration reaching the second ozone concentration level, controlling the ozone generator to output the ozone in the environment at a second time after the first time;
determining that a third ozone concentration has reached the first ozone concentration level;
controlling, based on the third ozone concentration reaching the first ozone concentration level, the ozone generator to stop outputting the ozone;
determining a second decay rate of the ozone during a second period of time;
determining a difference between the first decay rate and the second decay rate; and
performing an action based on the difference.

2. The system of claim 1, wherein the action comprises:
determining, based at least in part on the difference meeting or exceeding a threshold, that the environment is not disinfected; or
determining, based at least in part on the difference being below the threshold, that the environment is clean.

3. The system of claim 1, the operations further comprising sending a signal indicative of the difference to a remote computing device.

4. The system of claim 1, wherein the second period of time is after the first period of time.

5. The system of claim 1, the operations further comprising measuring one or more of: internal temperature of the environment, internal pressure of the environment, internal humidity of the environment, environment size, external temperature, external pressure, external humidity, or materials to be disinfected.

6. A method comprising:
controlling an ozone generator to output ozone in an environment to a first ozone concentration level at a first time;
determining a first decay rate of the ozone in the environment from the first ozone concentration level during a first period of time;
controlling the ozone generator to output the ozone in the environment to a second ozone concentration level at a second time;
determining a second decay rate of the ozone in the environment from the second ozone concentration level during a second period of time;
determining a difference between the first decay rate and the second decay rate; and
performing an action based on the difference.

7. The method of claim 6, further comprising controlling an air circulation device to facilitate the ozone to diffuse in the environment.

8. The method of claim 6, further comprising measuring the first ozone concentration level and the second ozone concentration level from multiple sensors in the environment.

9. The method of claim 8, wherein the multiple sensors include a first sensor and a second sensor, the method further comprising:
determining a sensor difference between a first reading of the first sensor and a second reading of the second sensor; and
sending an indication of the sensor difference to a remote computing device.

10. The method of claim 6, further comprising controlling at least one of an ultraviolet C (UVC) light unit or a humidifier unit based at least in part on the difference.

11. The method of claim 6, further comprising:
receiving a parameter from a remote computing device; and
controlling the ozone generator based at least in part on the parameter, wherein the parameter includes one or more of the first ozone concentration level, the second ozone concentration level, or an instruction for operating the ozone generator relative to a UVC light.

12. The method of claim 6, further comprising monitoring differences in decay rates of the ozone in the environment over a plurality of periods of time.

13. The method of claim 6, further comprising monitoring environmental data for regulatory compliance.

14. The method of claim 6, wherein the environment is an enclosed environment in which at least one of: the environment is to be disinfected or the environment contains an object to be disinfected.

15. One or more non-transitory computer-readable media storing instructions that, when executed, cause one or more processors to perform operations comprising:
controlling an ozone generator to output ozone in an environment to a first ozone concentration level at a first time;
determining a first decay rate of the ozone in the environment from the first ozone concentration level during a first period of time;
controlling the ozone generator to output the ozone in the environment to a second ozone concentration level at a second time;
determining a second decay rate of the ozone in the environment from the second ozone concentration level during a second period of time;
determining a difference between the first decay rate and the second decay rate; and
performing an action based on the difference.

16. The one or more non-transitory computer-readable media of claim 15, wherein the action comprises:
determining, based at least in part on the difference meeting or exceeding a threshold, that the environment is not disinfected; or
determining, based at least in part on the difference being below the threshold, that the environment is clean.

17. The one or more non-transitory computer-readable media of claim 15, further comprising sending a signal indicative of the difference to a remote computing device.

18. The one or more non-transitory computer-readable media of claim 15, wherein the second period of time is after the first period of time.

19. The one or more non-transitory computer-readable media of claim 15, the operations further comprising measuring one or more of: internal temperature of the environment, internal pressure of the environment, internal humidity of the environment, environment size, external temperature, external pressure, external humidity, or materials to be disinfected.

20. The one or more non-transitory computer-readable media of claim 15, the operations further comprising controlling a fan to circulate the ozone in the environment.
